(19) United States
(12) Patent Application Publication
(10) Pub. No.: US 2024/0294894 A1
(43) Pub. Date: Sep. 5, 2024

(54) IMPROVED BACILLUS HOST CELL WITH ALTERED REMA/REMB PROTEIN

(71) Applicant: BASF SE, Ludwigshafen (DE)

(72) Inventors: Max Fabian Felle, Ludwigshafen (DE); Mathis Appelbaum, Ludwigshafen (DE); Christopher Sauer, Ludwigshafen (DE); Stefan Janewein, Ludwigshafen (DE)

(21) Appl. No.: 18/572,942

(22) PCT Filed: Jun. 24, 2022
(86) PCT No.: PCT/EP2022/067442

§ 371 Date: Dec. 21, 2023

(30) Foreign Application Priority Data
Jun. 24, 2021 (EP) 21181347.2

(51) Int. Cl. C12N 9/54; C12N 15/75

(52) U.S. Cl. CPC C12N 9/54; C12N 15/75; C12Y 304/21062; C12N 2800/101

(57) ABSTRACT

The present invention relates to a Bacillus host cell for increased production of biological compounds. Specifically, the invention relates to a Bacillus host with genetic modifications in the remA and/or remB gene. The present invention further relates to a method for increased production of at least one polypeptide of interest based on cultivating the bacterial host cell of the present invention.

### FIELD OF THE INVENTION

The present invention relates to a Bacillus host cell for increased production of biological compounds. Specifically, the invention relates to a Bacillus host with genetic modifications in the remA and/or remB gene. The present invention further relates to a method for increased production of at least one compound of interest based on cultivating the bacterial host cell of the present invention.

### BACKGROUND

Microorganisms of the Bacillus genus are widely applied as industrial workhorses for the production of valuable compounds, such as chemicals, polymers and proteins, in particular proteins like washing- and/or cleaning-active enzymes or enzymes used for feed and food applications. The biotechnological production of these useful substances is conducted via fermentation of such Bacillus species and subsequent purification of the product. Bacillus species are capable of secreting significant amounts of protein to the fermentation broth. This allows a simple product purification process compared to intracellular production and explains the success of Bacillus in industrial application.

The production of biological compounds with Bacillus has been achieved by optimization the gene expression cassette. Promoters such as the aprE gene promoter (EP1244794), combinations of the PcryIIIA, PamyL and PamyQ promoters (WO994379, U.S. Pat. No. 5,955,310, WO2005098016), or the bacteriophage promoter PSPO1 (WO2015118126) driving high-level expression have been developed.

Likewise the mRNA stability of the resulting transcript has been optimized to increased half-life by introducing within the 5′UTR of the transcript stabilizing elements such the cryIIIA stabilizing element (WO9943835), the stabilizing element of the aprE gene (WO2016134213) and the stabilizing elements of the cotG, SP82, gsiB, grpE and rib genes (WO2008140615).

Furthermore, increasing the copy number of the expression cassette encoding the biological compound of interest has been realized to increase product yields. US20100248306 discloses a method for stable plasmid maintenance and WO15055558 stable and increased plasmid copy number within the cell. Various methods for stable integration of multiple polynucleotide copies within the chromosome of a cell have been successfully applied (US2003032186, US2008085535).

Bacterial production hosts have been genetically modified to remove undesired host cell proteins and improve product purity (WO2003093453) and enhance expression of a protein of interest (WO2003083125)

The optimization of the Bacillus host cell for the production biological compounds is of high relevance, where even small improvements in compound yield are significant in large scale industrial quantities. Therefore, the present invention relates to Bacillus host cells with increased biological compounds production capabilities.

### BRIEF SUMMARY OF THE INVENTION

It has been found in the studies underlying the present invention that a Bacillus host cell with genetic modifications in the remA and/or remB gene allows for an improved production of a compound of interest, in particular a polypeptide of interest, e.g. an exoenzyme, in said host cell. Accordingly, the present invention relates to a modified Bacillus host cell comprising an altered RemA protein and/or an altered RemB protein, wherein the Bacillus host cell is not a Bacillus subtilis cell.

In another preferred embodiment, the Bacillus host cell of the present invention comprises an expression cassette for the production of a compound of interest, preferably a polypeptide of interest. Thus, in another embodiment, the present invention refers to a method for producing a compound of interest, preferably a polypeptide of interest, comprising
a) providing a modified Bacillus host cell comprising an altered RemA protein and/or an altered RemB protein,
b) cultivating the host cell under conditions which allow for the expression of the compound of interest, and
c) optionally isolating the compound of interest from the cultivation medium.

Furthermore, the present invention refers to an altered RemA or RemB protein, which is of use for the generation of an improved Bacillus host cell, wherein the altered RemA protein comprises one or more non-conservative amino acid substitutions (as defined herein) at conserved amino acid positions of SEQ ID NO: 21, 25, 29, 33, or 37, preferably SEQ ID NO: 21, with an IC value equal or greater than 3.0, preferably equal or greater than 3.2, most preferably equal or greater than 3.5, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at amino acid position R18 and/or P29 of SEQ ID NO: 21 and wherein the altered RemB protein comprises one or more non-conservative amino acid substitutions (as defined herein) at conserved amino acid positions of SEQ ID NO: 23, 27, 31, 35, or 39, preferably, SEQ ID NO: 23, with an IC value equal or greater than 3.0, preferably equal or greater than 3.2, most preferably equal or greater than 3.5, preferably at one or more amino acid positions corresponding to amino acid positions selected from the group consisting of H4, G6, I19, K49, S50, Y59, S61, T67, L68, and R71, more preferably amino acid positions G6, I19, S62, T67, L68, and R71 of SEQ ID NO: 23, most preferably amino acid positions G6, T67, L68, and R71 of SEQ ID NO: 23.

### BRIEF DESCRIPTION OF THE DRAWINGS

FIG. 1: Multiple sequence alignment of the indicated RemA proteins. The protein sequence numbering is indicated for each single sequence and the consensus sequence. The alignment is shown as blocks of 10 sites. Amino acid changes different from the consensus sequence are indicated in bold letter.

FIG. 2: Multiple sequence alignment of the indicated RemB proteins. The protein sequence numbering is indicated for each single sequence and the consensus sequence. The alignment is shown as blocks of 10 sites. Amino acid changes different from the consensus sequence are indicated in bold letter.

### DETAILED DESCRIPTION OF THE INVENTION

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, and even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

The terms “polynucleotide”, “nucleic acid sequence”, “nucleotide sequence”, “nucleic acid”, “nucleic acid molecule” are used interchangeably herein and refer to nucleotides, typically deoxyribonucleotides, in a polymeric unbranched form of any length. The terms “polypeptide” and “protein” are used interchangeably herein and refer to amino acids in a polymeric form of any length, linked together by peptide bonds.

The terms “coding for” and “encoding” are used interchangeably herein. Typically, the terms refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein, if transcription and translation of mRNA corresponding to that gene produces the protein in a cell or other biological system.

For the purpose of the present invention, the term “modified”, “genetically modified”, or “genetic modification” (also called herein “recombinant” or “transgenic”) with regard to a cell or an organism means that the cell or organism contains a heterologous polynucleotide which is either obtained from a different organism or generated by man by gene technology. Hence, a modified cell is a non-native cell.

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “altered protein” as used herein refers to a protein that has been amended by man by gene technology and can be encoded by a modified endogenous gene or by an exogenous gene (also referred to as heterologous to the host cell), e.g., an exogenous gene encoding said protein inserted into a host cell, preferably, along with a deleted or inactivated endogenous gene encoding the unaltered protein. Hence, an altered protein is a non-native protein.

The term “nonsense mutation” is a point mutation that leads to a stop codon within the coding region of a protein-encoding sequence.

The term “missense mutation” is a point mutation that leads to another amino acid at the respective amino acid position.

The term “inactivating a gene” means that the expression of the gene has been reduced as compared to expression in a control cell. Preferably, expression of the gene in the bacterial host cell of the present invention has been reduced by at least 40% such as at least 50%, at least 60%, at least 70%, at least 80%, or at least 90% as compared to the corresponding expression in the control cell. More preferably, said expression has been reduced by at least 95%. Most preferably, it has been reduced by 100%, i.e. has been eliminated completely.

The inactivation of a gene as referred to herein may be achieved by any method deemed appropriate. In an embodiment, the gene has been inactivated by mutation, i.e. by mutating the gene. Preferably, said mutation is a deletion, preferably, said gene has been deleted.

As used herein, the “deletion of a gene” refers to the deletion of the entire coding sequence, deletion of part of the coding sequence, or deletion of the coding sequence including flanking regions with the end result being that the deleted gene is effectively non-functional. In simple terms, a “deletion” is defined as a change in either nucleotide or amino acid sequence in which one or more nucleotides or amino acid residues, respectively, have been removed (i.e., are absent). Thus, a deletion strain has fewer nucleotides or amino acids than the respective wild-type organism.

The term “inactivating a protein” means that the protein is altered in its amino acid sequence in a way that the function of the protein in the cell has been reduced as compared to the non-altered protein. Preferably, the function of the protein in the bacterial host cell of the present invention has been reduced by at least 10%, such as at least 40%, as at least 50%, at least 60%, at least 70%, at least 80%, or at least 90% as compared to the corresponding function of the non-altered protein. More preferably, said function has been reduced by at least 95%. Most preferably, the function has been reduced by 100%, i.e. the protein in completely non-functional. A “control cell” as referred to herein is a control cell of the same species which does not carry the respective modification, preferably which differs from the host cell only in that it does not carry the respective modification. Thus, the control cell is an unmodified cell, such as a wild-type cell, i.e. an unmodified wild-type cell, preferably a Bacillus licheniformis cell, which does not carry the respective modification. Preferably, the control cell is a Bacillus licheniformis cell, which differs from the host cell only in that it does not carry the respective modification.

### The Host Cell

The present invention is directed to a Bacillus host cell comprising an altered RemA protein and/or an altered RemB protein, preferably an altered RemA protein.

For example, the Bacillus host cell may be a Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus subtilis, Bacillus thuringiensis or Bacillus velezensis. In one embodiment, the bacterial host cell is a Bacillus amyloliquefaciens, Bacillus velezensis Bacillus lentus, Bacillus licheniformis, Bacillus pumilus, Bacillus stearothermophilus or Bacillus subtilis cell. In another embodiment, the bacterial host cell is a Bacillus licheniformis cell, Bacillus pumilus cell or a Bacillus subtilis cell, in a specifically preferred embodiment a Bacillus licheniformis cell.

In a preferred embodiment, the Bacillus host cell is not a Bacillus subtilis cell. In this embodiment, the Bacillus host cell is preferably selected from the group consisting of Bacillus amyloliquefaciens, Bacillus velezensis, Bacillus lentus, Bacillus licheniformis, Bacillus pumilus, and Bacillus stearothermophilus, preferably, selected from Bacillus licheniformis and Bacillus pumilus.

In a preferred embodiment, the host cell is a Bacillus licheniformis host cell. For example, the host cell may be a host cell of the Bacillus licheniformis strain ATCC14580 (which is the same as DSM13, see Veith et al. “The complete genome sequence of Bacillus licheniformis DSM13, an organism with great industrial potential.” J. Mol. Microbiol. Biotechnol. (2004) 7:204-211).

In another preferred embodiment, the host cell is a Bacillus velezensis host cell. For example, the host cell may be a host cell of the Bacillus velezensis strain FZB42.

In another preferred embodiment, the host cell is a Bacillus amyloliquefaciens host cell. For example, the host cell may be a host cell of the Bacillus amyloliquefaciens strain XH7.

In another preferred embodiment, the host cell is a Bacillus pumilus host cell. For example, the host cell may be a host cell of the Bacillus pumilus strain DSM27.

In another preferred embodiment, the host cell is a Bacillus lentus host cell. For example, the host cell may be a host cell of the Bacillus lentus strain DSM9.

In another preferred embodiment, the host cell is a Bacillus alcalophilus host cell. For example, the host cell may be a host cell of the Bacillus alcalophilus strain ATCC27647.

In another preferred embodiment, the host cell is a Bacillus methanolicus host cell. For example, the host cell may be a host cell of the Bacillus methanolicus strain PB1 (DSM16454) or Bacillus methanolicus strain MGA3 (ATCC53907).

The Bacillus host cell of the present invention shall be a modified host cell. Specifically, the Bacillus host comprises an altered RemA protein and/or an altered RemB protein, preferably an altered RemA protein. Thus, the Bacillus host comprises a variant of a native RemA protein, or the Bacillus host comprises a variant of a native RemB protein, preferably a variant of a native RemA protein. In particular preferred is a Bacillus host cell comprises an altered RemA protein with reduced RemA-function in the Bacillus host cell and/or an altered RemB protein with reduced RemB-function in the Bacillus host cell. Preferred is a Bacillus host cell that comprises an altered RemA protein has an inactivated RemA-function in the Bacillus host cell and/or an altered RemB protein which has an inactivated RemB-function in the Bacillus host cell. Thus, particularly preferred, the Bacillus host cell comprises an inactivated RemA protein and/or an inactivated RemB protein, preferably an inactivated RemA protein. Preferably, alteration of the RemA and/or RemB protein is an inactivation of the RemA and/or RemB protein in the Bacillus host cell. In this preferred embodiment, the altered RemA protein and/or altered RemB protein is still present, but the RemA protein and/or the RemB protein has an inactivated function, preferably no function.

Without being bound to theory, the present inventors believe that a reduction of function of the RemA and/or RemA protein in the Bacillus host cell leads to an increased production of a compound of interest by the Bacillus host cell. Thus, preferably, the host comprises an altered RemA protein, preferably wherein the alteration of the RemA protein confers a loss of RemA-mediated transcription activation. Preferably, the alteration of the RemA protein confers a reduced DNA binding affinity of the RemA protein. Further preferably, the Bacillus host comprises an altered RemB protein, preferably wherein the alteration of the RemB protein confers a loss of RemB-mediated transcription activation.

In one embodiment, the alteration of the RemA protein is caused by one or more point mutations, insertions, or partial deletions in the gene coding for the RemA protein. Preferably, the alteration of the RemA protein is caused by one or more point mutations in the gene coding for the RemA protein. Preferably, the one or more point mutations in the gene coding for the RemA protein are selected from the group consisting of missense mutations, nonsense mutation, and frame-shift mutations. Preferably, the one or more point mutations in the gene coding for the RemA protein is one or more missense mutations. Preferably, the one or more point mutations in the remA gene result in an inactivation of the RemA protein in the Bacillus host cell.

Preferably, the alteration of the RemB protein is caused by one or more point mutations, insertions, or partial deletions in the gene coding for the RemB protein. Preferably, the alteration of the RemB protein is caused by one or more point mutations in the gene coding for the RemB protein. Preferably, the one or more point mutations in the gene coding for the RemB protein are selected from the group consisting of missense mutations, nonsense mutations, and frame-shift mutations. Preferably, the one or more point mutations in the gene coding for the RemB protein is one or more missense mutations. Preferably, the one or more point mutations in the remB gene result in an inactivation of the RemB protein in the Bacillus host cell.

Preferably, the one or more missense point mutations in the gene coding for the RemA protein are at positions coding for conserved amino acids in the RemA protein. A conserved amino acid position in a protein can also be described as a position having an IC value equal or greater 2.0. The IC (Information Content) value as used herein is the computed value R_Sequence (I) as is described by Schneider, T. D.; Stephens, R. M. Sequence Logos: A New Way to Display Consensus Sequences. Nucleic Acids Res. 1990, 18 (20), 6097-6100, with using 20 states for amino acid sequences. Preferably, the one or more missense point mutations in the gene coding for the RemA protein are at positions coding conserved amino acid positions of SEQ ID NO: 21, 25, 29, 33, or 37, preferably SEQ ID NO: 21, with an IC value equal or greater than 2.0, preferably equal or greater than 2.5, more preferably equal or greater than 3.0, or even more preferably equal or greater than 3.2, most preferably equal or greater than 3.5. Preferably, the one or more missense point mutations in the gene coding for the RemA protein are at positions coding for conserved amino acid positions of SEQ ID NO: 21 with an IC value equal or greater than 3.2, most preferably equal or greater than 3.5.

Preferably, the one or more point mutations in the gene coding for the RemA protein result in non-conservative amino acid substitutions (as defined herein, see, e.g., Table 7) in the RemA protein. Thus, preferably the altered RemA protein comprises one or more non-conservative amino acid exchanges. Preferably the altered RemA protein comprises one or more non-conservative amino acid exchanges that lead to a reduced function of the RemA protein in the Bacillus cell. Preferably the altered RemA protein comprises one or more non-conservative amino acid exchanges that lead to an inactivation of the RemA protein in the Bacillus cell. Preferably, the one or more point mutations in the gene coding for the RemA protein result in non-conservative amino acid substitutions, preferably inactivating substitutions, at conserved amino acid positions of SEQ ID NO: 21, 25, 29, 33, or 37, preferably SEQ ID NO: 21, with an IC value equal or greater than 2.0, preferably equal or greater than 2.5, more preferably equal or greater than 3.0, or even more preferably equal or greater than 3.2, most preferably equal or greater than 3.5. Preferably, the one or more missense point mutations in the gene coding for the RemA protein are at positions coding for conserved amino acid positions of SEQ ID NO: 21 with an IC value equal or greater than 3.2, most preferably equal or greater than 3.5. Preferably, the one or more point mutations in the gene coding for the RemA protein result in non-conservative amino acid substitutions, preferably as shown in Table 7, preferably inactivating substitutions, at conserved amino acid positions of SEQ ID NO: 21 with an IC value equal or greater than 3.0, or even more preferably equal or greater than 3.2, most preferably equal or greater than 3.5.

Preferably, the one or more point mutations in the gene coding for the RemA protein result in non-conservative amino acid substitutions, preferably inactivating substitutions, at one or more of amino acid positions corresponding to amino acid positions 5-77 of SEQ ID NO: 21, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at one or more amino acid position selected from amino acid positions corresponding to R18 and P29 of SEQ ID NO: 21.

Preferably the altered RemA protein comprises at least one of the substitutions X18W and X29S at amino acid position R18 and P29 of SEQ ID NO: 21. Preferably the altered RemA protein comprises the substitutions X18W and X29S at amino acid position R18 and P29 of SEQ ID NO: 21.

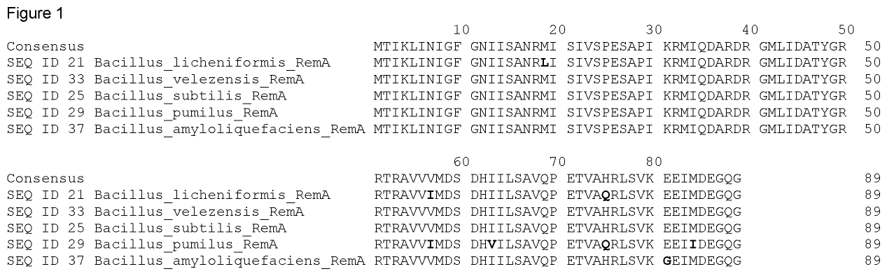

The term “amino acid positions corresponding to amino acid positions” followed by certain amino acid positions indicated by number or residue and number of SEQ ID NO: 21 shall mean that for referring to certain amino acid positions in a particular RemA protein a sequence alignment is made with SEQ ID NO: 21 as displayed in FIG. 1 and the amino acid numbering of SEQ ID NO: 21 at a certain amino acid position is used for reference (i.e., according to the numbering of SEQ ID NO: 21), e.g., in SEQ ID NO: 29 (RemA of Bacillus pumilus) M84 of SEQ ID NO: 21 (RemA of Bacillus licheniformis) would correspond to 184 of SEQ ID NO: 29.

Preferably, the altered RemA protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21, 25, 29, 33, or 37, preferably SEQ ID NO: 21. Preferably, the altered RemA protein comprises an amino acid sequence having at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21, 25, 29, 33, or 37, preferably SEQ ID NO: 21.

Preferably, the altered RemA protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21, 25, 29, 33, or 37, preferably SEQ ID NO: 21 and one or more amino acid substitutions, preferably one or more non-conservative amino acid substitutions, preferably inactivating substitutions, at one or more of amino acid positions corresponding to amino acid positions 5-77 of SEQ ID NO: 21, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at one or more, preferably both, amino acid position selected from amino acid positions corresponding to R18 and P29 of SEQ ID NO: 21. Preferably, the altered RemA protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21 and one or more non-conservative amino acid substitutions, preferably inactivating substitutions, at one or more of amino acid positions corresponding to amino acid positions 5-77 of SEQ ID NO: 21, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at one or more amino acid position selected from amino acid positions corresponding to R18 and P29 of SEQ ID NO: 21.

Preferably the altered RemA protein comprises at least one of the substitutions X18W and X29S at amino acid position R18 and P29 of SEQ ID NO: 21. Preferably the altered RemA protein comprises the substitutions X18W and X29S at amino acid position R18 and P29 of SEQ ID NO: 21. Preferably the altered RemA protein comprises an amino acid sequence having at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21 and comprises at least one, preferably both, of the substitutions R18W and P29S at amino acid position R18 and P29 of SEQ ID NO: 21.

Preferably, the one or more point mutations in the gene coding for the RemB protein result in non-conservative amino acid substitutions (as defined herein, see, e.g., Table 7). Thus, preferably the altered RemB protein comprises one or more non-conservative amino acid exchanges.

Preferably the altered RemB protein comprises one or more non-conservative amino acid exchanges that lead to a reduced function of the RemB protein in the Bacillus cell. Preferably the altered RemB protein comprises one or more non-conservative amino acid exchanges that lead to an inactivation of the RemB protein in the Bacillus cell. Preferably, the one or more point mutations in the gene coding for the RemB protein result in non-conservative amino acid substitutions, preferably inactivating substitutions, at conserved amino acid positions of SEQ ID NO: 23, 25, 29, 35, or 39, preferably SEQ ID NO: 23, with an IC value equal or greater than 3.0, or even more preferably equal or greater than 3.2, most preferably equal or greater than 3.5. Preferably, the one or more missense point mutations in the gene coding for the RemB protein are at positions coding for conserved amino acid positions of SEQ ID NO: 23 with an IC value equal or greater than 3.2, most preferably equal or greater than 3.5. Preferably, the one or more missense point mutations in the gene coding for the RemB protein are at positions coding for conserved amino acid positions of SEQ ID NO: 23 with an IC value equal or greater than 3.2, most preferably equal or greater than 3.5. Preferably, the one or more point mutations in the gene coding for the RemB protein result in non-conservative amino acid substitutions, preferably as shown in Table 7, preferably inactivating substitutions, at conserved amino acid positions of SEQ ID NO: 23 with an IC value equal or greater than 3.0, or even more preferably equal or greater than 3.2′, most preferably equal or greater than 3.5.

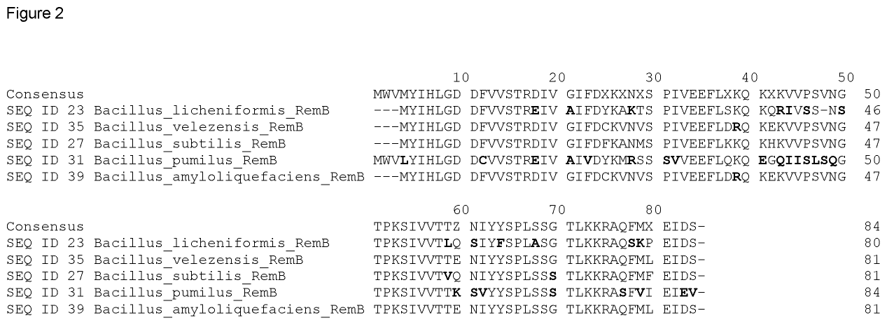

Preferably, the one or more missense point mutations in the gene coding for the RemB protein are at positions coding for conserved amino acids in the RemB protein, preferably at one or more of amino acid positions corresponding to amino acid positions 4-71 of SEQ ID NO: 23, preferably the one or more point mutations in the gene coding for the RemB protein result in non-conservative amino acid substitutions, preferably inactivating substitutions, at conserved amino acid positions of SEQ ID NO: 23, 27, 31, 35, or 39, preferably, SEQ ID NO: 23, with an IC value equal or greater than 3.0, preferably equal or greater than 3.2, most preferably equal or greater than 3.5, preferably at one or more amino acid positions corresponding to amino acid positions selected from the group consisting of H4, G6, I19, K49, S50, Y59, S61, T67, L68, and R71 of SEQ ID NO: 23, preferably at one or more amino acid positions selected from amino acid positions corresponding to G6, I19, S62, T67, L68, and R71 of SEQ ID NO: 23, more preferably at one or more amino acid positions selected from amino acid positions corresponding to G6, T67, L68, and R71 of SEQ ID NO: 23. The term “amino acid positions corresponding to amino acid positions” followed by certain amino acid positions indicated by number or residue and number of SEQ ID NO: 23 shall mean that for referring to certain amino acid positions in a particular RemB protein a sequence alignment is made with SEQ ID NO: 23 as displayed in FIG. 2 and the amino acid numbering of SEQ ID NO: 23 at a certain amino acid position is used for reference (i.e., according to the numbering of SEQ ID NO: 23), e.g., in SEQ ID NO: 31 (RemB of Bacillus pumilus) S80 of SEQ ID NO: 23 (RemB of Bacillus licheniformis) would correspond to V84 of SEQ ID NO: 31.

Preferably, the altered RemB protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 23, 27, 31, 35, or 39, preferably, SEQ ID NO: 23. Preferably, the RemB protein has at least 80%, preferably at least 90%, sequence identity to SEQ ID NO: 23, 27, 31, 35, or 39, preferably, SEQ ID NO: 23. Preferably, the altered RemB protein comprises an amino acid sequence having at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 23, 27, 31, 35, or 39, preferably, SEQ ID NO: 23.

Preferably, the altered RemB protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 23, 27, 31, 35, or 39, preferably, SEQ ID NO: 23 and one or more amino acid substitutions, preferably one or more substitutions at positions coding for conserved amino acids in the RemB protein, preferably at one or more of amino acid positions corresponding to amino acid positions 4-71 of SEQ ID NO: 23, 27, 31, 35, or 39, preferably, SEQ ID NO: 23, preferably the one or more substitutions in the RemB protein are non-conservative amino acid substitutions, preferably inactivating substitutions, preferably at one or more amino acid positions corresponding to amino acid positions selected from the group consisting of H4, G6, I19, K49, S50, Y59, S61, T67, L68, and R71 of SEQ ID NO: 23, preferably at one or more amino acid positions selected from amino acid positions corresponding to G6, I19, S62, T67, L68, and R71 of SEQ ID NO: 23, more preferably at one or more amino acid positions selected from amino acid positions corresponding to G6, T67, L68, and R71 of SEQ ID NO: 23.

Preferably, the altered RemB protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 23 and one or more substitutions at positions coding for conserved amino acids in the RemB protein, preferably at one or more of amino acid positions corresponding to amino acid positions 4-71 of SEQ ID NO: 23, preferably non-conservative amino acid substitutions, preferably inactivating substitutions, preferably at one or more amino acid positions corresponding to amino acid positions selected from the group consisting of H4, G6, I19, K49, S50, Y59, S61, T67, L68, and R71 of SEQ ID NO: 23, preferably at one or more amino acid positions selected from amino acid positions corresponding to G6, I19, S62, T67, L68, and R71 of SEQ ID NO: 23, more preferably at one or more amino acid positions selected from amino acid positions corresponding to G6, T67, L68, and R71 of SEQ ID NO: 23.

The altered RemA protein and/or RemB protein can be obtained in the Bacillus host cell by modifying the endogenous remA and/or remB gene and/or by introducing an exogenous gene coding for an altered RemA and/or RemB protein. In case of the latter, the endogenous remA and/or remB gene is preferably inactivated, preferably deleted. The exogenous gene coding for the altered RemA protein and/or RemB protein can be present in the host cell as expression plasmid or can be integrated into the genomic DNA of the host cell. In case of the latter, the integration of the exogenous gene coding for the altered RemA protein and/or RemB protein is preferably at the genomic location of the endogenous gene coding for the RemA and/or RemB protein and thereby deleting the endogenous RemA and/or RemB protein. Alternatively, the integration of the exogenous gene coding for the altered RemA protein and/or RemB protein under the control of a suitable promoter sequence is at a different genomic location, such as the amylase, protease aprE, or levansucrase sacB locus. Appropriate promoters for expressing the altered RemA and/or RemB protein in the Bacillus host cell are well-known in the art and described elsewhere herein in more detail. Preferably, the modified Bacillus host cell comprising the altered RemA protein and/or an altered RemB protein, which is newly introduced into the Bacillus host cell by a respective coding sequence, comprises a deletion of the endogenous RemA and/or RemB gene. Thus, preferably, the modified Bacillus host cell does not comprise a functional gene coding for the endogenous RemA and/or endogenous RemB protein.

The nucleic acid construct introduced into the Bacillus host cell, which encodes the altered RemA and/or RemB protein, can comprise a nucleic acid sequence coding for a RemA and/or RemB protein being derived from the same or from a different Bacillus species. Preferably, the altered RemA and/or RemB protein introduced into the Bacillus host cell is from the same Bacillus species, preferably from Bacillus licheniformis.

Thus, in one embodiment the present invention is directed to a method of producing the modified Bacillus host cell comprising the altered RemA protein and/or altered RemB protein. Preferably, the modified Bacillus host cell can be obtained by a method comprising the steps of
a) providing a Bacillus cell, preferably a Bacillus licheniformis cell, and
b) modifying the Bacillus cell provided under a) by modifying the endogenous gene coding for the endogenous RemA and/or endogenous RemB protein to code for an altered RemA and/or an altered RemB protein as described herein and thereby obtaining the modified Bacillus host cell.

In another embodiment, the modified Bacillus host cell can be obtained by a method comprising the steps of
a) providing a Bacillus cell, preferably a Bacillus licheniformis cell,
b) modifying the Bacillus cell provided under a) by introducing a nucleic acid construct comprising a gene coding for an altered RemA and/or an altered RemB protein as described herein, preferably, derived from the native Bacillus licheniformis RemA and/or RemB protein, preferably under the control of a suitable promoter sequence, into the Bacillus cell and thereby obtaining the modified Bacillus host cell, and
c) optionally inactivating, preferably deleting, the endogenous gene coding for the endogenous RemA and/or the endogenous RemB protein.

Also described herein, the Bacillus host cell can also comprise a deletion or inactivation of the endogenous remA gene or a deletion or inactivation of the endogenous remB gene. Thus, in one embodiment the invention refers to a Bacillus host cell comprising a deletion or inactivation of the endogenous remA gene and a deletion or inactivation of the endogenous remB gene. In another embodiment, the Bacillus host cell comprises an altered RemA protein as described herein and a deletion or inactivation of the endogenous remB gene. In a further embodiment, the Bacillus host cell comprises an altered RemB protein as described herein and a deletion or inactivation of the endogenous remA gene.

In one embodiment, the Bacillus host cell is used for producing a compound of interest as described elsewhere herein. The compound of interest can be endogenous or heterologous to the host cell. Preferably, the compound of interest is a protein, preferably an enzyme. Preferably the compound of interest is heterologous to the host cell. Preferably, the compound of interest is a protein, preferably an enzyme, heterologous to the host cell.

In one embodiment, the host cell comprises an expression cassette for the production of a compound of interest, preferably a polypeptide of interest. In one embodiment, the polypeptide of interest is an enzyme, such as an enzyme selected from the group consisting of amylase, protease, lipase, phospholipase, mannanase, phytase, xylanase, lactase, phosphatase, glucoamylase, nuclease, galactosidase, endoglucanase and cellulase, preferably a protease and/or an amylase.

Preferably, the modified Bacillus host cell comprising an altered RemA protein and/or an altered RemB protein comprises an increased production of the compound of interest, preferably compared to a Bacillus control cell that does not comprise the altered RemA protein and/or the altered RemB protein. Preferably, the modified Bacillus host cell, preferably the Bacillus licheniformis host cell, comprising an altered RemA protein and/or an altered RemB protein comprises an increased production of the protein of interest, preferably an enzyme, compared to a Bacillus host cell, preferably a Bacillus licheniformis control cell, that does not comprise the altered RemA protein and/or the altered RemB protein.

The terms “increased” and “enhanced” are used interchangeably herein and shall mean in the sense of the application preferably an increase of at least 10%, at least 20%, at least 25%, at least 30%, at least 40%, at least 50%, at least 60%, at least 70%, at least 80%, at least 90% or 100%.

The terms “decreased” and “reduced” are used interchangeably herein and shall mean in the sense of the application preferably a reduction of at least 10%, at least 20%, at least 25%, at least 30%, at least 40%, at least 50%, at least 60%, at least 70%, at least 80%, at least 90% or 95%. In some embodiments, the level of a gene product or its activity is reduced by 100%.

Thus, the activity is eliminated completely. This may be achieved by inactivating the gene.

Methods for the generation of modified Bacillus cells and altered proteins, e.g., by introduction of foreign nucleic acids, chromosomal gene deletion, substitution, mutation, and inactivation, are known in the art.

The introduction of DNA into a host cell, in one embodiment a Bacillus cell, may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). Specific transformation protocols are known in the art for various types of host cells (see, e.g., for E. coli protoplast transformation see Hanahan, 1983, J. Mol. Biol. 166: 557-580). Gene inactivation can be achieved by homologous recombination, i.e. an incoming DNA molecule comprises sequences that are homologous to the 5′ and 3′ flanking sequences of the target sequence on the chromosome of the host cell (e.g. Bacillus) to be inactivated. Subsequently the sequence between said flanking sequences is replaced by the homologous sequences of the incoming DNA molecule in the process of homologous recombination, i.e. the sequence is deleted from the chromosome. Likewise “gene integration”, i.e. a DNA sequence such as a gene expression cassette with or without a selectable marker, can be integrated into the chromosome of the bacterial host cell by homologous recombination. Hence. The DNA sequence to be integrated is flanked by DNA sequences that are homologous to the 5′ and 3′ flanking sequences on the chromosome. It is understood in terms of the invention that gene integration can also combine gene integration and gene deletion in one step, i.e. a DNA sequence on the chromosome is replaced by the incoming DNA sequence for gene integration.

Homologous recombination can be achieved by two different methods known in the art: By two consecutive rounds of homologous recombination (Campbell recombination) with circular plasmid DNA, e.g. based on the well-known temperature sensitive plasmid pE194 (Nahrstedt et al., Strain development in Bacillus licheniformis: construction of biologically contained mutants deficient in sporulation and DNA repair. J Biotechnol. 2005 Sep. 29; 119(3):245-54).

The integration of the deletion plasmid containing an incoming DNA molecule comprising sequences that are homologous to the 5′ and 3′ flanking sequences of the target sequence on the chromosome is achieved by a first homologous recombination (Campbell recombination) with the first homologous region under selective conditions for the selectable marker and cultivation at the non-permissive temperature, i.e. that blocks plasmid replication. The second homologous recombination with the second homologous region is achieved by removal of selective pressure and cultivation at the permissive temperature, i.e. plasmid replication takes place, resulting in excision of the plasmid from the chromosome.

Alternatively, a non-replicative ‘suicide’ plasmid can be used forcing the integration by selection on the selectable marker. Only cells that have integrated the plasmid into the genome by homologous recombination are able to grow under the selective conditions. Plasmid removal/excision from the chromosome is achieved with a second homologous recombination which is forced by the activation of a counterselection marker present on the plasmid.

The second method of homologous recombination refers to two homologous recombination events simultaneously taking place, also known as ‘double crossing over’ or ‘double homologous recombination. The incoming DNA sequence is linear and can be obtained by PCR, linearization of plasmid DNA or preparation of chromosomal DNA which inevitable results in fragmented linear DNA. WO0308125 uses linear DNA constructs (either linearized plasmids or PCR fragments) comprising a selectable marker flanked by the 5′ and 3′ homologous regions which are used for genomic integration via double crossing over homologous recombination. It is well understood that next to the selectable marker additional DNA, such as gene expression cassettes, when flanked by said homologous region are integrated into the chromosome of the bacterial host cell.

Homologous recombination requires DNA sequences homologous to the 5′ and 3′ flanking sequences of the target sequence on the chromosome of the host cell of sufficient size, hence should contain a sufficient number of nucleic acid such as 100 to 1,500 base pairs, preferably 400 to 1,500 base pairs, and most preferably 800 to 1,500 base pairs, which have a high degree of identity to the corresponding target sequence to enhance the probability of homologous recombination (Dubnau, 1993, Genetic exchange and homologous recombination. In Bacillus subtilis and Other Gram-positive Bacteria, p. 555-584. Edited by A. I. Sonenshein, J. A. Hoch & R. Losick, Washington DC, American Society for Microbiology; Michel and Ehrlich, 1984, The EMBO Journal, vol. 3, pp. 2879-2884).

Gene inactivation by deletion/insertion/substitution can also be achieved by CRISPR/Cas9 genome editing technologies where the CRISPR cutting properties could be used to disrupt genes in almost any organism's genome with unprecedented ease (Mali P, et al (2013) Science. 339(6121):819-823; Cong L, et al (2013) Science 339(6121)). Recently it became clear that providing a template for repair, e.g. homologous regions, allowed for editing the genome with nearly any desired sequence at nearly any site, transforming CRISPR into a powerful gene editing tool (WO/2014/150624, WO/2014/204728).

CRISPR-based genome editing systems for application in gram positive organisms have been well described such as the Bacillus species based single-plasmid system approach, i.e. comprising the Cas9 endonuclease, the gRNA (e.g. sgRNA or crRNA/tracrRNA), repair homology sequences (donor DNA) on one single E. coli-Bacillus shuttle vector (Altenbuchner, (2016): Applied and environmental microbiology 82 (17), 5421-5427; Zhou, et al. (2019): International journal of biological macromolecules 122, 329-337), or dual plasmid system or with Cas9 endonuclease integrated into the Bacillus genome as described e.g. in WO2020206202 and WO2020206197.

Alternatively to “directed” methods of inactivation it is understood in the scope of the invention that whole-cell mutagenesis by applying mutagenizing conditions such as exposure of the cells to UV radiation, or chemical mutagenizing chemicals such as NTG (N-methyl-N′-nitro-N-nitrosoguanidine), EMS (ethyl-methane-sulfonate), in combination with screening and/-or selection of the desired property, e.g. reduced lipase/esterase activity is a well-known approach to achieve functional inactivation.

Further, a gene may have been inactivated by gene silencing. Gene silencing can be achieved by introducing into said bacterial host cell antisense expression constructs that result in antisense RNAs complementary to the mRNA of the gene, thereby inhibiting expression of said genes. Alternatively, the expression of said gene can be inhibited by blocking transcriptional initiation or transcriptional elongation through the mechanism of CRISPR-inhibition (WO18009520).

### The Altered RemA and RemB Protein and Nucleic Acids Encoding the Same

In another embodiment, the present invention is directed to an altered RemA protein or an altered RemB protein and to a nucleic acid encoding the altered RemA protein or the altered RemB protein as described herein, preferably directed to an altered RemA protein and to a nucleic acid encoding the altered RemA protein as described herein. Preferably, the altered RemA protein is a variant of a native RemA protein. Preferably, the altered RemB protein is a variant of a native RemB protein. Preferably, the altered RemA protein and/or the RemB protein has reduced function in the Bacillus host cell. Preferably, the alteration of the RemA and/or RemB protein leads to an inactivated function of the RemA and/or RemB protein in the Bacillus host cell. Thus, the present invention refers to a variant of a RemA and/or RemB protein, preferably an inactivated RemA and/or inactivated RemB protein. Preferably, the altered RemA or RemB protein comprises one or more amino acid exchanges. Preferably, the altered RemA or RemB protein comprises one or more non-conservative amino acid substitutions (preferably as shown in Table 7), preferably at conserved amino acid positions.

Preferably, the one or more point mutations in the gene coding for the RemA protein are at conserved amino acid positions of SEQ ID NO: 21, 25, 29, 33, or 37, preferably SEQ ID NO: 21, with an IC value equal or greater than 3.0, more preferably equal or greater than 3.2, most preferably equal or greater than 3.5. Thus, the present invention is also directed to a nucleic acid molecule encoding such altered RemA protein comprising one or more point mutations in triplets coding for conserved amino acid positions of SEQ ID NO: 21, 25, 29, 33, or 37, preferably SEQ ID NO: 21, with an IC value equal or greater than 3.0, preferably equal or greater than 3.2, most preferably equal or greater than 3.5.

Preferably, the one or more point mutations in the gene coding for the RemA protein result in non-conservative amino acid substitutions (as defined herein, see, e.g., Table 7)) in the RemA protein. Thus, preferably the altered RemA protein comprises one or more non-conservative amino acid exchanges. Thus, preferably the altered RemA protein comprises one or more non-conservative amino acid exchanges that lead to a reduced function of the RemA protein in the Bacillus cell. Preferably the altered RemA protein comprises one or more non-conservative amino acid exchanges that lead to an inactivation of the RemA protein in the Bacillus cell. Preferably, the one or more point mutations in the gene coding for the RemA protein result in non-conservative amino acid substitutions, preferably inactivating substitutions, at conserved amino acid positions of SEQ ID NO: 21, 25, 29, 33, or 37, preferably SEQ ID NO: 21, with an IC value equal or greater than 3.0, more preferably equal or greater than 3.2, most preferably equal or greater than 3.5.

Preferably, the one or more point mutations in the gene coding for the RemA protein result in non-conservative amino acid substitutions, preferably inactivating substitutions, at one or more of amino acid positions corresponding to amino acid positions 5-77 of SEQ ID NO: 21, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at amino acid position R18 and/or P29 of SEQ ID NO: 21. Preferably, the altered RemA protein comprises an amino acid sequence having at least 60%, at least 70%, at least 80%, at least 90%, at least 91%, at least 92%, at least 93%, at least 94%, at least 95%, at least 96%, at least 97%, at least 98%, or at least 99%, but less than 100% identity to SEQ ID NO: 21, 25, 29, 33, or 37, preferably SEQ ID NO: 21. Preferably, the altered RemA protein comprises an amino acid sequence having at least 80%, at least 90%, at least 91%, at least 92%, at least 93%, at least 94%, at least 95%, at least 96%, at least 97%, at least 98%, or at least 99%, but less than 100% identity to SEQ ID NO: 21.

Preferably, the one or more missense point mutations in the gene coding for the RemB protein are at positions coding for conserved amino acids in the RemB protein. Preferably, the one or more point mutations in the gene coding for the RemB protein are at conserved amino acid positions of SEQ ID NO: 23, 27, 31, 35, or 39, preferably, SEQ ID NO: 23, with an IC value equal or greater than 3.0, more preferably equal or greater than 3.2, most preferably equal or greater than 3.5. Thus, the present invention is also directed to a nucleic acid molecule encoding such altered RemB protein comprising one or more point mutations in triplets coding for conserved amino acid positions of SEQ ID NO: 23, 27, 31, 35, or 39, preferably, SEQ ID NO: 23, with an IC value equal or greater than 3.0, more preferably equal or greater than 3.2, most preferably equal or greater than 3.5.

Preferably, the one or more point mutations in the gene coding for the RemB protein result in non-conservative amino acid substitutions (as defined herein). Thus, preferably the altered RemB protein comprises one or more non-conservative amino acid exchanges. Thus, preferably the altered RemB protein comprises one or more non-conservative amino acid exchanges that lead to a reduced function of the RemB protein in the Bacillus cell. Preferably the altered RemB protein comprises one or more non-conservative amino acid exchanges that lead to an inactivation of the RemB protein in the Bacillus cell. Preferably, the one or more point mutations in the gene coding for the RemB protein result in non-conservative amino acid substitutions, preferably inactivating substitutions, at conserved amino acid positions of SEQ ID NO: 23, 27, 31, 35, or 39, preferably, SEQ ID NO: 23, with an IC value equal or greater than 3.0, preferably equal or greater than 3.2, most preferably equal or greater than 3.5. Preferably, the one or more missense point mutations in the gene coding for the RemB protein are at positions coding for conserved amino acids in the RemB protein, preferably at one or more of amino acid positions corresponding to amino acid positions 4-71 of SEQ ID NO: 23, 27, 31, 35, or 39, preferably, SEQ ID NO: 23, preferably the one or more point mutations in the gene coding for the RemB protein result in non-conservative amino acid substitutions, preferably inactivating substitutions, at conserved amino acid positions of SEQ ID NO: 23, 27, 31, 35, or 39, preferably, SEQ ID NO: 23, with an IC value equal or greater than 3.0, preferably equal or greater than 3.2, most preferably equal or greater than 3.5, preferably at one or more amino acid positions corresponding to amino acid positions selected from the group consisting of H4, G6, I19, K49, S50, Y59, S61, T67, L68, and R71, preferably at one or more amino acid positions selected from amino acid positions corresponding to G6, I19, S62, T67, L68, and R71 of SEQ ID NO: 23, more preferably at one or more amino acid positions selected from amino acid positions corresponding to G6, T67, L68, and R71 of SEQ ID NO: 23. Preferably, the RemB protein has at least 80%, preferably at least 90%, sequence identity to SEQ ID NO: 23, 27, 31, 35, or 39, preferably, SEQ ID NO: 23. Preferably, the altered RemB protein comprises an amino acid sequence having at least 60%, at least 70%, at least 80%, at least 90%, at least 91%, at least 92%, at least 93%, at least 94%, at least 95%, at least 96%, at least 97%, at least 98%, or at least 99%, but less than 100% identity to SEQ ID NO: 23, 27, 31, 35, or 39, preferably, SEQ ID NO: 23. Preferably, the altered RemB protein comprises an amino acid sequence having at least 80%, at least 90%, at least 91%, at least 92%, at least 93%, at least 94%, at least 95%, at least 96%, at least 97%, at least 98%, or at least 99%, but less than 100% identity to SEQ ID NO: 23.

In another embodiment, the present invention refers to a nucleic acid or nucleic acid construct encoding the altered RemA and/or altered RemB protein as described herein. Preferably, the nucleic acid or nucleic acid construct encoding the altered RemA protein comprises:
(a) a polynucleotide encoding an amino acid sequence having at least 80%, at least 85%, at least 90%, at least 91%, at least 92%, at least 93%, at least 94%, at least 95%, at least, 96%, at least 97%, at least 98%, or at least 99%, but less than 100% sequence identity to SEQ ID NO: 21, 25, 29, 33, or 37, preferably SEQ ID NO: 21, 25, 29, 33, or 37, preferably SEQ ID NO: 21;
(b) a polynucleotide having at least 80%, at least 85%, at least 90%, at least 91%, at least 92%, at least 93%, at least 94%, at least 95%, at least, 96%, at least 97%, at least 98%, or at least 99%, but less than 100% sequence identity with SEQ ID NO: 20, 24, 28, 32, or 36, preferably SEQ ID NO: 20,
(c) a polynucleotide that hybridizes under high stringency conditions with the complement of a polynucleotide shown in SEQ ID NO: 20, 24, 28, 32, or 36, preferably SEQ ID NO: 20,
(d) a polynucleotide that having at least 95%, but less than 100% sequence identity to SEQ ID NO: 20, 24, 28, 32, or 36, preferably SEQ ID NO: 20, wherein the polynucleotide further differs to SEQ ID NO: 20, 24, 28, 32, or 36, preferably SEQ ID NO: 20, merely by the degeneration of the genetic code, or
(e) a fragment of (a), (b), (c), or (d).

Preferably, the nucleic acid or nucleic acid construct encoding the altered RemB protein comprises:
(a) a polynucleotide encoding an amino acid sequence having at least 80%, at least 85%, at least 90%, at least 91%, at least 92%, at least 93%, at least 94%, at least 95%, at least, 96%, at least 97%, at least 98%, or at least 99%, but less than 100% sequence identity to SEQ ID NO: 23, 27, 31, 35, or 39, preferably, SEQ ID NO: 23;
(b) a polynucleotide having at least 80%, at least 85%, at least 90%, at least 91%, at least 92%, at least 93%, at least 94%, at least 95%, at least, 96%, at least 97%, at least 98%, or at least 99%, but less than 100% sequence identity with SEQ ID NO: 22, 26, 30, 34, or 38, preferably, SEQ ID NO: 22,
(c) a polynucleotide that hybridizes under high stringency conditions with the complement of a polynucleotide shown in SEQ ID NO: 22, 26, 30, 34, or 38, preferably, SEQ ID NO: 22,
(d) a polynucleotide that having at least 95%, but less than 100% sequence identity to SEQ ID NO: 22, 26, 30, 34, or 38, preferably, SEQ ID NO: 22, wherein the polynucleotide further differs to SEQ ID NO: 22, 26, 30, 34, or 38, preferably, SEQ ID NO: 22, merely by the degeneration of the genetic code, or
(e) a fragment of (a), (b), (c), or (d).

The altered RemA protein described herein is a variant of a parent RemA protein. The altered RemB protein described herein is a variant of a parent RemB protein.

A variant of a parent protein may have an amino acid sequence which has a certain percent identity to the amino acid sequence of the parent sequence. Thus, a variant of a parent polypeptide may comprise an amino acid sequence which is at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, but below 100% identical to an amino acid sequence of the parent polypeptide. Variants may be, thus, defined by their sequence identity when compared to a parent polypeptide. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percent-identity applies:
%-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. For non-protein-coding DNA sequences the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

For nucleic acids, similar sequences can also be determined by hybridization using respective stringency conditions. The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C. The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

In one embodiment, a variant polypeptide comprises 1-30, 1-20, 1-10, or 1-5 amino acid substitutions, preferably, such substitutions are not pertaining to the functional domains of an enzyme.

Variants may be defined by their sequence similarity when compared to a parent polypeptide. Sequence similarity usually is provided as “% sequence similarity” or “%-similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is referred to as “conservative mutation”. Polypeptide variants comprising conservative mutations appear to have a minimal effect on protein folding resulting in certain polypeptide, preferably enzyme, properties being substantially maintained when compared to the polypeptide properties of the parent polypeptide.

For determination of %-similarity according to this invention the following applies, which is also in accordance with the BLOSUM62 matrix (matrix (Henikoff, J. G.; Proc. Natl. Acad. Sci. USA 89, 10915-10919 (1992)), which is one of the most used amino acids substitution matrix for database searching and sequence alignments. An amino acid exchange is defined as similar if the value of the BLOSUM62 substitution matrix for the pair of letters is positive. Table 6 shows conservative exchanges.

### Table 6:

Amino acid A is similar to amino acid S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acid S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining to the functional domains of an enzyme. In another embodiment conservative mutations are not pertaining to the catalytic centers of an enzyme.

Therefore, according to the present invention the following calculation of percent-similarity applies:
%-similarity=[(identical residues+similar residues)/length of the alignment region which is showing the respective sequence of this invention over its complete length]*100. Thus, sequence similarity in relation to comparison of two amino acid sequences herein is calculated by dividing the number of identical residues plus the number of similar residues by the length of the alignment region which is showing the respective sequence of this invention over its complete length. This value is multiplied by 100 to give “%-similarity”.

Especially, variant polypeptide comprising conservative mutations which are at least m percent similar to the respective parent sequences with m being an integer between 50 and 100, preferably 50, 55, 60, 65, 70, 75, 80, 85, 90, 91, 92, 93, 94, 95, 96, 97, 98 or 99 compared to the full length polypeptide sequence, are expected to have essentially unchanged polypeptide properties. In another embodiment conservative mutations are not pertaining to the catalytic centers of an enzyme. In one embodiment, a variant polypeptide comprises 1-30, 1-20, 1-10, or 1-5 conservative amino acid substitutions, preferably, such substitutions are not pertaining to the functional domains of an enzyme.

Likewise, the exchange of one amino acid with a non-similar amino acid is referred to as “non-conservative mutation”. Enzyme variants comprising non-conservative mutations appear to have an effect on protein folding resulting in certain enzyme properties being different when compared to the enzyme properties of the parent enzyme. Hence, an amino acid exchange is defined as non-conservative if the value of the BLOSUM62 substitution matrix for the pair of letters is negative. Table 7 shows non-conservative exchanges.

### Table 7:

Amino acid A is non-similar to amino acids D, E, F, H, I, K, L, M, N, P, Q, R, W, Y
Amino acid C is non-similar to amino acids D, E, F, G, H, I, K, L, M, N, P, Q, R, S, T, V, W, Y
Amino acid D is non-similar to amino acids A, C, F, G, H, I, K, L, M, P, R, T, V, W, Y
Amino acid E is non-similar to amino acids A, C, F, G, I, L, M, P, T, V, W, Y
Amino acid F is non-similar to amino acids A, C, D, E, G, H, K, N, P, Q, R, S, T, V
Amino acid G is non-similar to amino acids C, D, E, F, H, I, K, L, M, P, Q, R, T, V, W, Y
Amino acid H is non-similar to amino acids A, C, D, F, G, I, K, L, M, P, S, T, V, W
Amino acid I is non-similar to amino acids A, C, D, E, G, H, K, N, P, Q, R, S, T, W, Y
Amino acid K is non-similar to amino acids A, C, D, F, G, H, I, L, M, P, T, V, W, Y
Amino acid L is non-similar to amino acids A, C, D, E, G, H, K, N, P, Q, R, S, T, W, Y
Amino acid M is non-similar to amino acids A, C, D, E, G, H, K, N, P, R, S, T, W, Y
Amino acid N is non-similar to amino acids A, C, F, I, L, M, P, V, W, Y
Amino acid P is non-similar to amino acids A, C, D, E, F, G, H, I, K, L, M, N, Q, R, S, T, V, W, Y
Amino acid Q is non-similar to amino acids A, C, F, G, I, L, P, T, V, W, Y
Amino acid R is non-similar to amino acids A, C, D, F, G, I, L, M, P, S, T, V, W, Y
Amino acid S is non-similar to amino acids C, F, H, I, L, M, P, R, V, W, Y
Amino acid T is non-similar to amino acids C, D, E, F, G, H, I, K, L, M, P, Q, R, W, Y
Amino acid V is non-similar to amino acids C, D, E, F, G, H, K, N, P, Q, R, S, W, Y
Amino acid W is non-similar to amino acids A, C, D, E, G, H, I, K, L, M, N, P, Q, R, S, T, V
Amino acid Y is non-similar to amino acids A, C, D, E, G, I, K, L, M, N, P, Q, R, S, T, V

### The Method for Producing a Compound of Interest

In another embodiment, the present invention refers to a method for producing a compound of interest, preferably a polypeptide of interest. For producing the polypeptide of interest, the modified Bacillus host cell shall comprise at least one polynucleotide encoding the polypeptide of interest, wherein said polynucleotide is operably linked to a promoter. Accordingly, the host cell shall comprise an expression cassette for at least one polypeptide of interest.

Thus, the present invention relates to a method for producing a compound of interest, preferably a polypeptide of interest, comprising
a) providing the modified Bacillus host cell comprising an altered RemA protein and/or an altered RemB protein as described herein,
b) introducing onto said modified Bacillus host cell an expression cassette for a compound of interest, preferably a polypeptide of interest,
b) cultivating the host cell under conditions which allow for the expression of the compound of interest, and
c) optionally isolating the compound of interest from the cultivation medium.

Preferably, the Bacillus host cell for the method for producing a compound of interest is selected from the group consisting of Bacillus pumilus, Bacillus velezensis, Bacillus amyloliquefaciens, Bacillus alcalophilus, Bacillus licheniformis, Bacillus paralicheniformis, Bacillus lentus, Bacillus clausii, Bacillus halodurans, Bacillus megaterium, Bacillus methanolicus, Geobacillus stearothermophilus (Bacillus stearothermophilus), Bacillus mojavensis, Bacillus globigii, or Bacillus subtilis. For example, the Bacillus host cell for the method for producing a compound of interest is belongs to the species Bacillus pumilus, Bacillus velezensis, Bacillus amyloliquefaciens, Bacillus licheniformis or Bacillus subtilis. In another preferred embodiment, the host cell is a Bacillus subtilis host cell. For example, the host cell may be a host cell of the Bacillus subtilis strain NCIB 3610. However, in one embodiment, the host cell for the method for producing a compound of interest is not a Bacillus subtilis host cell. Thus, for the method for producing a compound of interest preferably the host cell is selected from the group consisting of Bacillus pumilus, Bacillus velezensis, Bacillus amyloliquefaciens, Bacillus alcalophilus, Bacillus licheniformis, Bacillus paralicheniformis, Bacillus lentus, Bacillus clausii, Bacillus halodurans, Bacillus megaterium, Bacillus methanolicus, Geobacillus stearothermophilus (Bacillus stearothermophilus), Bacillus mojavensis, or Bacillus globigii. Preferably the host cell for the method for producing a compound of interest is selected from the group consisting of Bacillus pumilus, Bacillus velezensis, Bacillus amyloliquefaciens, or Bacillus licheniformis.

The explanations and definitions given herein above in connection with the modified host cell of the present invention apply mutatis mutandis to the method of the present invention.

The term “cultivating” as used herein refers to keeping alive and/or propagating the modified host cell comprised in a culture at least for a predetermined time. The term encompasses phases of exponential cell growth at the beginning of growth after inoculation as well as phases of stationary growth. The cultivation conditions shall allow for the expression, i.e. the production, of the polypeptide of interest. Such conditions can be chosen by the skilled person without further ado. Exemplary conditions for the cultivation of the modified host cell are described in Example 3. In an embodiment of the method of the present invention, the cultivation in step b) is carried out as fed batch cultivation.

The method of the present invention, if applied, allows for increasing the expression, i.e. the production, of the at least one compound, preferably polypeptide, of interest. Preferably, expression is increased as compared to the expression in an unmodified control cell. In a preferred embodiment, expression of the at least one polypeptide of interest is increased by at least 10%, 20% or by at least 40%, such as by at least 50%, or at least 80% as compared to the expression in the control cell. For example, expression of the at least one polypeptide of interest may be increased by 20% to 100%, such as by 40% to 60%, as compared to the control cell. Further, it is envisaged that the expression is increased by at least 100%, 150%, 200%. 250% or 300%, such as by 200% to 300%. Typically, the expression can be measured by determining the amount of the compound of interest in the host cell and/or in the cultivation medium.

In one embodiment, the expression cassette for the expression of the compound of interest in the Bacillus host cell is heterologous to the Bacillus host cell. Preferably, the polynucleotide encoding at least one polypeptide of interest is heterologous to the Bacillus host cell. Preferably, in one embodiment, the polynucleotide encoding at least one polypeptide of interest is heterologous to the bacterial host cell. The term “heterologous” (or exogenous or foreign or recombinant or non-native) polypeptide or protein as used throughout the specification is defined herein as a polypeptide or protein that is not native to the host cell. Similarly, the term “heterologous” (or exogenous or foreign or recombinant or non-native) polynucleotide refers to a polynucleotide that is not native to the host cell.

In an embodiment, the at least one polynucleotide encoding a polypeptide of interest is present on a plasmid. The term “plasmid” refers to an extrachromosomal circular DNA, i.e. a vector that is autonomously replicating in the host cell. Thus, a plasmid is understood as extrachromosomal vector.

In a preferred embodiment, the replication of a plasmid shall be independent of the replication of the chromosome of the bacterial host cell. For autonomous replication, the expression vector may further comprise an origin of replication enabling the vector to replicate autonomously in the host cell in question. Bacterial origins of replication include but are not limited to the origins of replication of plasmids pBR322, pUC19, pSC101, pACYC177, and pACYC184 permitting replication in E. coli (Sambrook, J. and Russell, D. W. Molecular cloning. A laboratory manual, 3rd ed, Cold Spring Harbor Laboratory Press, Cold Spring Harbor, N Y. 2001; Cohen, S. N., Chang, A. C. Y., Boyer, H. W., & Helling, R. B. (1973). Construction of Biologically Functional Bacterial Plasmids In Vitro. Proceedings of the National Academy of Sciences of the United States of America, 70(11), 3240-3244), and pUB110, pC194, pE194, pTB19, pAMß1, and pTA1060 permitting replication in Bacillus (Janniere, L., Bruand, C., and Ehrlich, S. D. (1990). Structurally stable Bacillus subtilis cloning vectors. Gene 87, 53-6; Ehrlich, S. D., Bruand, C., Sozhamannan, S., Dabert, P., Gros, M. F., Janniere, L., and Gruss, A. (1991). Plasmid replication and structural stability in Bacillus subtilis. Res. Microbiol. 142, 869-873), and pE194 (Dempsey, L. A. and Dubnau, D. A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436).

The copy number of a plasmid is defined as the average number of plasmids per bacterial cell or per chromosome under normal growth conditions. Moreover, there are different types of replication origins that result in different copy numbers in the bacterial host. The plasmid replicon pBS72 (accession number AY102630.1) and the plasmids pTB19 and derivatives pTB51, pTB52 confer low copy number with 6 copies and 1 to 8 copies, respectively, within Bacillus cells whereas plasmids pE194 (accession number V01278.1) and pUB110 (accession number M19465.1)/pBC16 (accession number U32369.1) confer low-medium copy number with 14-20 and medium copy number with 30-50 copies per cell, respectively. Plasmid pE194 was analyzed in more detail (Villafane, et al (1987): J. Bacteriol. 169(10), 4822-4829) and several pE194-cop mutants described having high copy numbers within Bacillus ranging from 85 copies to 202 copies. Moreover, plasmid pE194 is temperature sensitive with stable copy number up to 37° C., however abolished replication above 43° C. In addition, it exists a pE194 variant referred to as pE194ts with two point mutations within the cop-repF region (nt 1235 ad nt 1431) leading to a more drastic temperature sensitivity—stable copy number up to 32° C., however only 1 to 2 copies per cell at 37° C.

In one embodiment, the vectors contain one or more selectable markers that permit easy selection of transformed cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO91/09129, where the selectable marker is on a separate vector.

In another embodiment, the at least one polynucleotide encoding a polypeptide of interest is stably integrated into the bacterial chromosome.

### Promoter

The at least one polynucleotide encoding a polypeptide of interest shall be operably linked to a promoter.

The term “operably linked” as used herein refers to a functional linkage between the promoter sequence and the polynucleotide encoding a polypeptide of interest, such that the promoter sequence is able to initiate transcription of the polynucleotide encoding a polypeptide of interest (herein also referred to as gene of interest).

A “promoter” or “promoter sequence” is a nucleotide sequence located upstream of a gene on the same strand as the gene that enables that gene's transcription. A promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors.

The person skilled in the art is capable to select suitable promoters for expressing the third alanine racemase and the polypeptide of interest. For example, the polynucleotide encoding the polypeptide of interest is, preferably, operably linked to an “inducer-dependent promoter” or an “inducer-independent promoter”. Further, the polynucleotide encoding the third alanine racemase is, preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule, the presence of which in the fermentation medium is capable of affecting an increase in expression of a gene by increasing the activity of an inducer-dependent promoter operably linked to the gene. Preferably, the inducer molecule is a carbohydrate or an analog thereof. In one embodiment, the inducer molecule is a secondary carbon source of the Bacillus cell. In the presence of a mixture of carbohydrates, cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| | Regulator | Type | | |
|---|---|---|---|---|
| Operon | a) | b) | Inducer | Organism |
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β- | B. subtilis |
| | | | glucosides | |
| levDEFG sacL | LevR | A | fructose | B. subtilis |
| mtlAD | MtlR | A | mannitol | B. subtilis |
| manPA-yjdF | ManR | A | mannose | B. subtilis |
| manR | ManR | A | mannose | B. subtilis |
| bglFB bglG | BglG | AT | β-glucosides | E. coli |
| lacTEGF | LacT | AT | lactose | L. casei |
| lacZYA | lacI | R | Allolactose; | E. coli |
| | | | IPTG (Isopropyl | |
| | | | β-D-1-thiogalactopy- | |
| | | | ranoside) | |
| araBAD | araC | AR | L-arabinose | E. coli |
| xylAB | XyIR | R | Xylose | B. subtilis |

a: transcriptional regulator protein
b: A: activator
AT: antiterminator
R: repressor
AR: activator/repressor

In contrast thereto, the activity of promoters that do not depend on the presence of an inducer molecule (herein called ‘inducer-independent promoters’) are either constitutively active or can be increased regardless of the presence of an inducer molecule that is added to the fermentation medium.

Constitutive promoters are independent of other cellular regulating factors and transcription initiation is dependent on sigma factor A (sigA). The sigA-dependent promoters comprise the sigma factor A specific recognition sites ‘-35’-region and ‘-10’-region.

Preferably, the ‘inducer-independent promoter’ sequence is selected from the group consisting of constitutive promoters not limited to promoters Pveg, PlepA, PserA, PymdA, Pfba and derivatives thereof with different strength of gene expression (Guiziou et al, (2016): Nucleic Acids Res. 44(15), 7495-7508), the aprE promoter of Subtilisin encoding aprE gene of Bacilli, the bacteriophage SPO1 promoters P4, P5, P15 (WO15118126), the cryIIIA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably an aprE promoter sequence.

WO9102792 discloses the functionality of the promoter of the alkaline protease gene for the large-scale production of subtilisin Carlsberg-type protease in Bacillus licheniformis and its production in a fermentation process.

The promoters of the Bacillus pumilus genes aprE1 and aprE2 encoding for Subtilisin proteases have been applied for the expression of recombinant protease and amylase in Bacillus pumilus (Küppers T, Wiechert W. Microb Cell Fact. 2014 Mar. 24; 13(1):46.). In particular the PaprE1-III promoter variant comprising nucleotides nt-382 relative to the start ATG showed very high productivity compared to PaprE1-IV promoter variant (nt-357 relative to the start ATG).

An “aprE promoter”, “aprE-type promoter” or “aprE promoter sequence” is the nucleotide sequence (or parts or variants thereof) located upstream of an aprE gene, i.e., a gene coding for a Bacillus subtilisin Carlsberg protease, on the same strand as the aprE gene that enables that aprE gene's transcription. The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

WO9102792 discloses the functionality of the promoter of the alkaline protease gene for the large-scale production of subtilisin Carlsberg-type protease in Bacillus licheniformis. In particular, WO9102792 describes the 5′ region of the subtilisin Carlsberg protease encoding aprE gene of Bacillus licheniformis (FIG. 27) comprising the functional aprE gene promoter and the 5′UTR comprising the ribosome binding site (Shine Dalgarno sequence).

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

Further optionally the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

With respect to the 5′UTR the invention in particular teaches to combine the promoter of the present invention with a 5′UTR comprising one or more stabilizing elements. This way the mRNAs synthesized from the promoter region may be processed to generate mRNA transcript with a stabilizer sequence at the 5′ end of the transcript. Preferably such a stabilizer sequence at the 5′end of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis CryIIIA mRNA stabilising sequence or bacteriophage SP82 mRNA stabilising sequence, more preferably a mRNA stabilising sequence according to SEQ ID NO. 4 or 5 of WO08140615, more preferably a modified mRNA stabilising sequence according to SEQ ID NO. 6 of WO08140615, or fragments of these sequences which maintain the mRNA stabilizing function.

Preferred mRNA stabilizing elements are selected from the group consisting of aprE, grpE, cotG, SP82, RSBgsiB, CryIIIA mRNA stabilizing elements, or according to fragments of these sequences which maintain the mRNA stabilizing function. A preferred mRNA stabilizing element is the grpE mRNA stabilizing element (corresponding to SEQ ID NO. 2 of WO08148575).

The 5′UTR also preferably comprises a modified rib leader sequence located downstream of the promoter and upstream of a ribosome binding site (RBS). In the context of the present invention a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5′-region of the rib operon between the transcription start and the translation start codon of the first gene in the operon, ribG. Suitable rib leader sequences are described in WO2015/1181296, in particular pages 23-25, incorporated herein by reference.

For industrial fermentation processes, the bacterial host cell may be genetically modified to meet the needs of highest product purity and regulatory requirements. It is therefore in scope of the invention to use Bacillus production hosts that may additionally contain modifications, e.g., deletions or disruptions, of other genes that may be detrimental to the production, recovery or application of a polypeptide of interest. In one embodiment, a bacterial host cell is a protease-deficient cell. The bacterial host cell, e.g., Bacillus cell, preferably comprises a disruption or deletion of extracellular protease genes including but not limited to aprE, mpr, vpr, bpr, and/or epr. Further preferably the bacterial host cell does not produce spores. Further preferably the bacterial host cell, e.g., a Bacillus cell, comprises a disruption or deletion of genes involved in sporulation. Genes involved in sporulation are well known in the art (EP1391502), comprising but not limited to sigE, sigF, spoIIGA, spoIIE, sigG, spoIVCB, yqfD. In a preferred embodiment, the sigF gene is deleted. Further, preferably the bacterial host cell, e.g., Bacillus cell, comprises a disruption or deletion of one of the genes involved in the biosynthesis of surfactin, e.g., srfA, srfB, srfC, and/or srfD, see, for example, U.S. Pat. No. 5,958,728. It is also preferred that the bacterial host cell comprises a disruption or deletion of one of the genes involved in the biosynthesis of polyglutamic acid (US2016002591). Accordingly, at least one gene involved in poly-gamma-glutamate (pga) production has been inactivated (such as deleted). Preferably, the at least one gene involved in poly-gamma-glutamate (pga) is at least one gene selected from ywsC (pgsB), ywtA (pgsC), ywtB (pgsA) and ywtC (pgsE). Preferably, all aforementioned genes, i.e. ywsC (pgsB), ywtA (pgsC), ywtB (pgsA) and ywtC (pgsE) have been inactivated (such as deleted). Other genes, including but not limited to the amyE gene, which are detrimental to the production, recovery or application of a polypeptide of interest may also be disrupted or deleted.

In one embodiment, the Bacillus cell comprises a selectable marker. The selectable marker can be antibiotic resistance markers such as ampicillin, kanamycin, erythromycin, chloramphenicol or tetracycline, or an auxotrophic resistance marker.

Optionally the Bacillus cell might comprise a counterselection markers as described herein. In a preferred embodiment, the counterselection polypeptide is a polypeptide which involved in the pyrimidine metabolism. Thus, the counterselection polypeptide, such as oroP, pyrE, pyrF, upp, uses flourated analogons of intermediates in the pyrmidine metabolism, such as, 5-fluoro-orotate or 5-fluoro-uridine. Alternatively, toxins of toxin-anti-toxin systems (TA) such as the mazEF, ccdAB could be used as functional counterselection polypeptides in Bacillus (see Dong, H., Zhang, D. Current development in genetic engineering strategies of Bacillus species. Microb Cell Fact 13, 63 (2014)). In an even more preferred embodiment, the couterselection polypeptide is a cytosine deaminase, such as provided by the codBA system (Kostner D, Rachinger M, Liebl W, Ehrenreich A. Markerless deletion of putative alanine dehydrogenase genes in Bacillus licheniformis using a codBA-based counterselection technique. Microbiology. 2017; 163(11): 1532-1539). Preferably, the counterselection agent is 5-fluoro-cytosine.

### The Compound of Interest

The host cell of the present invention shall further comprise an expression cassette for the production of a compound of interest, preferably a polypeptide of interest.

Compounds of interest maybe polymers, preferably hyaluronic acidy, preferably as described in (WO2005098016), or polyglutamic acid, preferably as described in EP2196534, or maybe vitamins, preferably vitamin B5, preferably as described in WO2010018169, or riboflavin, preferably as described in WO2017036903, or may be polypeptides, preferably enzymes.

The term “polypeptide of interest” as used herein refers to any protein, peptide or fragment thereof which is intended to be produced in the bacterial host cell. A protein, thus, encompasses polypeptides, peptides, fragments thereof as well as fusion proteins and the like.

Preferably, the compound of interest, preferably polypeptide of interest, is secreted by the Bacillus host cell.

Preferably, the polypeptide of interest is an enzyme, such as an exoenzyme. An exoenzyme (or extracellular enzyme), is an enzyme that is secreted by the host cell.

In a particularly preferred embodiment, the enzyme is classified as an oxidoreductase (EC 1), a transferase (EC 2), a hydrolase (EC 3), a lyase (EC 4), an isomerase (EC 5), or a ligase (EC 6). In a preferred embodiment, the protein of interest is an enzyme suitable to be used in detergents, feed and food applications.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1), beta-beta amylase (EC 3.2.1.2), a cellulase (EC 3.2.1.4), an endo-1,3-beta-xylanase xylanase (EC 3.2.1.32), an endo-1,4-beta-xylanase (EC 3.2.1.8), a lactase (EC 3.2.1.108), a galactosidase (EC 3.2.1.23 and EC 3.2.1.24), a mannanase (EC 3.2.1.24 and EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, ß-galactosidase, lactase glucoamylase, nuclease, and cellulase, preferably, amylase, mannanase, lactase or protease, preferably, an amylase and a protease. Most preferred is a serine protease (EC 3.4.21), preferably a subtilisin protease.

In particular, the following proteins of interest are preferred:

Enzymes having proteolytic activity are called “proteases” or “peptidases”. Proteases are active proteins exerting “protease activity” or “proteolytic activity”. Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endo-peptidases of unknown catalytic mechanism (EC 3.4.99). Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coro-nase®, Coronase® Ultra, Neutrase®, Everlase® and Esperase® (Novozymes A/S), those sold under the tradename Maxatase®, Maxacal®, Maxapem®, Purafect®, Purafect® Prime, Pura-fect MA®, Purafect Ox®, Purafect OxP®, Puramax®, Properase®, FN2®, FN3®, FN4®, Ex-cellase®, Eraser®, Ultimase®, Opticlean®, Effectenz®, Preferenz® and Optimase® (Dan-isco/DuPont), Axapem™ (Gist-Brocases N. V.), Bacillus lentus Alkaline Protease, and KAP (Bacillus alkalophilus subtilisin) from Kao. At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”. Preferably, the protease is a protease variant of Bacillus lentus alkaline protease (BLAP), most preferably BLAP comprising the substitution R101E (according to BPN′ numbering). Proteases according to the invention have proteolytic activity. The methods for determining proteolytic activity are well-known in the literature (see e.g. Gupta et al. (2002), Appl. Microbiol. Bio-technol. 60: 381-395).

Thus, the present invention relates to a method for producing an enzyme, preferably a protease or an amylase, comprising
a) providing the modified Bacillus host cell comprising an altered RemA protein and/or an altered RemB protein as described herein,
b) introducing onto said modified Bacillus host cell an expression cassette for an enzyme, preferably a protease or an amylase,
b) cultivating the host cell under conditions which allow for the expression of the enzyme, and
c) optionally isolating the enzyme from the cultivation medium.

### PREFERRED EMBODIMENTS

1. A modified Bacillus host cell comprising an altered RemA protein and/or an altered RemB protein, wherein the Bacillus host cell is not a Bacillus subtilis cell.

2. The modified Bacillus host cell of embodiment 1, wherein the alteration of the RemA protein is caused by one or more point mutations, preferably one or more missense mutations, in the gene coding for the RemA protein and wherein the alteration of the RemB protein is caused by one or more point mutations, preferably one or more missense mutations, in the gene coding for the RemB protein.

3. The modified Bacillus host cell of any one of the preceding embodiments, wherein the altered RemA protein and/or altered RemB Protein comprises one or more non-conservative mutations at conserved amino acid positions caused by one or more missense mutations.

4. The modified Bacillus host cell of any one of the preceding embodiments, wherein the altered RemA protein comprises one or more non-conservative mutations, preferably at conserved amino acid positions, that lead to a deactivation of the RemA protein in the Bacillus host cell.

5. The modified Bacillus host cell of any one of the preceding embodiments, wherein the altered RemB protein comprises one or more non-conservative mutations, preferably at conserved amino acid positions, that lead to a deactivation of the RemB protein in the Bacillus host cell.

6. The modified Bacillus host cell of any one of the preceding embodiments, wherein the one or more missense point mutations in the gene coding for the RemA protein are at positions coding for conserved amino acids in the RemA protein, preferably at one or more of amino acid positions corresponding to amino acid positions 5-77 of SEQ ID NO: 21.

7. The modified Bacillus host cell of any one of the preceding embodiments, wherein the one or more point mutations in the gene coding for the RemA protein result in non-conservative amino acid substitutions at conserved amino acid positions of SEQ ID NO: 21, 25, 29, 33, or 37, preferably SEQ ID NO: 21, with an IC value equal or greater than 3.0, preferably equal or greater than 3.2, most preferably equal or greater than 3.5, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at amino acid position R18 and/or P29 of SEQ ID NO: 21.

8. The modified Bacillus host cell of any one of the preceding embodiments, wherein the one or more point mutations in the gene coding for the RemA protein result in at least one of the substitutions X18W and X29S at amino acid position R18 and P29 of SEQ ID NO: 21.

9. The modified Bacillus host cell of any one of the preceding embodiments, wherein the one or more missense point mutations in the gene coding for the RemB protein are at positions coding for conserved amino acids in the RemB protein, preferably at one or more of amino acid positions corresponding to amino acid positions 4-71 of SEQ ID NO: 23.

10. The modified Bacillus host cell of any one of the preceding embodiments, wherein the one or more point mutations in the gene coding for the RemB protein result in non-conservative amino acid substitutions at conserved amino acid positions of SEQ ID NO: 23, 25, 29, 33, or 37, preferably SEQ ID NO: 21, with an IC value equal or greater than 3.0, preferably equal or greater than 3.2, most preferably equal or greater than 3.5, preferably at one or more amino acid positions corresponding to amino acid positions selected from the group consisting of H4, G6, I19, K49, S50, Y59, S61, T67, L68, and R71, preferably at one or more amino acid positions selected from amino acid positions corresponding to G6, I19, S62, T67, L68, and R71 of SEQ ID NO: 23, more preferably at one or more amino acid positions selected from amino acid positions corresponding to G6, T67, L68, and R71 of SEQ ID NO: 23.

11. The modified Bacillus host cell of any one of the preceding embodiments, wherein the alteration of the RemA and/or RemB protein is an inactivation of the RemA and/or RemB protein.

12. The modified Bacillus host cell of any one of the preceding embodiments, wherein the host cell belongs to the species Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus thuringiensis or Bacillus velezensis, preferably Bacillus licheniformis.

13. The modified Bacillus host cell of any of the preceding embodiments, wherein the altered RemA protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 21, 25, 29, 33, or 37, preferably SEQ ID NO: 21, and wherein the altered RemB protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 23, 27, 31, 35, or 39, preferably, SEQ ID NO: 23.

14. The modified Bacillus host cell of any of the preceding embodiments, wherein the altered RemA protein has least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21, 25, 29, 33, or 37, preferably SEQ ID NO: 21, and comprises at least one, preferably both, of the substitutions X18W and X29S at amino acid position R18 and P29 of SEQ ID NO: 21, preferably wherein the altered RemA protein has least 80%, at least 85%, at least 90%, at least 91%, at least 92%, at least 93%, at least 94%, at least 95%, at least 96%, at least 97%, at least 98%, or at least 99%, but less than 100% identity to SEQ ID NO: 21 and comprises at least one, preferably both, of the substitutions R18W and P29S at amino acid position R18 and P29 of SEQ ID NO: 21.

15. The modified Bacillus host cell of any of the preceding embodiments, wherein the Bacillus host cell comprises a deletion or inactivation of the endogenous remA and/or endogenous remB gene.

16. A modified Bacillus host cell comprising an altered RemA protein, wherein the alteration of the RemA protein is due to one or more missense point mutations in the gene coding for the RemA protein at positions coding for conserved amino acids in the RemA protein, preferably at one or more of amino acid positions corresponding to amino acid positions 5-77 of SEQ ID NO: 21, wherein the Bacillus host cell is not a Bacillus subtilis cell, wherein the alteration of the RemA protein is an inactivation of the RemA protein, and wherein the altered RemA protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 21, 25, 29, 33, or 37, preferably SEQ ID NO: 21.

17. The modified Bacillus host cell of embodiment 16, wherein the altered RemA protein in the modified Bacillus host cell is encoded by an exogenous gene introduced into the Bacillus host cell.

18. The modified Bacillus host cell of any of embodiment 16 or 17, wherein the Bacillus host cell comprises a deletion or inactivation of the endogenous remA gene.

19. The modified Bacillus host cell of any of embodiments 16-18, wherein the Bacillus host cell comprise an altered RemB protein as described herein.

20. The modified Bacillus host cell of any of embodiments 16-19, wherein the Bacillus host cell comprise a deletion or inactivation of the endogenous remB gene.

21. A modified Bacillus host cell comprising an altered RemB protein, wherein the alteration of the RemA protein is due to one or more missense point mutations in the gene coding for the RemA protein at positions coding for conserved amino acids in the RemB protein, preferably at one or more of amino acid positions corresponding to amino acid positions 5-77 of SEQ ID NO: 23, wherein the Bacillus host cell is not a Bacillus subtilis cell, wherein the alteration of the RemB protein is an inactivation of the RemB protein, and wherein the RemA protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 23, 27, 31, 35, or 39, preferably SEQ ID NO: 23.

22. The modified Bacillus host cell of embodiment 21, wherein the altered RemB protein in the modified Bacillus host cell is encoded by an exogenous gene introduced into the Bacillus host cell.

23. The modified Bacillus host cell of any of embodiments 21 or 22, wherein the Bacillus host cell comprises a deletion or inactivation of the endogenous remB gene.

24. The modified Bacillus host cell of any of embodiments 21-23, wherein the Bacillus host cell comprise an altered RemA protein as described herein.

25. The modified Bacillus host cell of any of embodiments 21-24, wherein the Bacillus host cell comprise a deletion or inactivation of the endogenous remA gene.

26. The modified Bacillus host cell of any of the preceding embodiments, wherein the modified Bacillus host cells is a Bacillus licheniformis host cell.

27. The modified Bacillus host cell of any one of the preceding embodiments, wherein the host cell comprises an expression cassette for the production of a compound of interest, preferably a polypeptide of interest.

28. The modified Bacillus host cell of embodiment 27, wherein the polypeptide of interest is an enzyme, such as an enzyme selected from the group consisting of amylase, protease, lipase, phospholipase, mannanase, phytase, xylanase, lactase, phosphatase, glucoamylase, nuclease, galactosidase, endoglucanase and cellulase, preferably a protease.

29. The modified Bacillus host cell of any of embodiments 27 or 28, wherein the modified Bacillus host cell comprises an increased production of the compound of interest compared to a Bacillus control cell that does not comprise the altered RemA protein and/or the altered RemB protein.

30. The modified Bacillus host cell of any of the preceding embodiment, wherein the modified Bacillus host cell comprises an altered RemA protein as described herein and a deleted endogenous RemB protein or wherein the modified Bacillus host cell comprises an altered RemB protein as described herein and a deleted endogenous RemA protein.

31. A method for producing a compound of interest, preferably a polypeptide of interest, comprising
a) providing a modified Bacillus host cell comprising an altered RemA protein and/or an altered RemB protein as described herein,
b) cultivating the host cell under conditions which allow for the expression of the compound of interest, and
c) optionally isolating the compound of interest from the cultivation medium.

32. A method for increasing the producing a compound of interest, preferably a polypeptide of interest, by a Bacillus host cell comprising
a) providing a modified Bacillus host cell comprising an altered RemA protein and/or an altered RemB protein as described herein,
b) cultivating the host cell under conditions which allow for the expression of the compound of interest, and
c) optionally isolating the compound of interest from the cultivation medium.

33. A method for producing an enzyme, preferably a protease or an amylase, comprising
a) providing a modified Bacillus host cell comprising an altered RemA protein and/or an altered RemB protein as described herein, preferably, wherein the altered RemA protein and/or altered RemB Protein comprises one or more non-conservative mutations, preferably deactivating mutations, at conserved amino acid positions,
b) cultivating the host cell under conditions which allow for the expression of the compound of interest, and
c) optionally isolating the compound of interest from the cultivation medium.

34. The method for producing a compound of interest of any of embodiments 31-33, wherein the Bacillus host cell is selected from the group consisting of Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus subtilis, Bacillus thuringiensis or Bacillus velezensis, preferably Bacillus licheniformis.

35. A altered RemA or RemB protein, wherein the altered RemA protein comprises one or more non-conservative amino acid substitutions at conserved amino acid positions of SEQ ID NO: 21 with an IC value equal or greater than 3.0, preferably equal or greater than 3.2, most preferably equal or greater than 3.5, preferably at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, most preferably at amino acid position R18 and/or P29 of SEQ ID NO: 21 and wherein the altered RemB protein comprises one or more non-conservative amino acid substitutions at conserved amino acid positions of SEQ ID NO: 23, 27, 31, 35, or 39, preferably, SEQ ID NO: 23, with an IC value equal or greater than 3.0, preferably equal or greater than 3.2, most preferably equal or greater than 3.5, preferably at one or more amino acid positions corresponding to amino acid positions selected from the group consisting of H4, G6, I19, K49, S50, Y59, S61, T67, L68, and R71, more preferably amino acid positions G6, I19, S62, T67, L68, and R71 of SEQ ID NO: 23, most preferably amino acid positions G6, T67, L68, and R71 of SEQ ID NO: 23.

36. The altered RemA protein and/or the altered RemB of embodiment 35, wherein the non-conservative amino acid substitutions at conserved amino acid positions are substitutions that reduce the function of the RemA protein and/or the RemB protein in the Bacillus cell.

37. The altered RemA protein of any of embodiments 35 or 36, wherein the RemA protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 21.

38. The altered RemB protein of any of embodiments 35 or 36, wherein the RemB protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 23, 27, 31, 35, or 39, preferably, SEQ ID NO: 23.

39. Use of an altered RemA protein and/or an altered RemB protein as described herein for increasing the production of a compound of interest by a Bacillus cell, wherein the Bacillus host cell is not a Bacillus subtilis cell.

### EXAMPLES

### Materials and Methods

The following examples only serve to illustrate the invention. The numerous possible variations that are obvious to a person skilled in the art also fall within the scope of the invention.

Unless otherwise stated the following experiments have been performed by applying standard equipment, methods, chemicals, and biochemicals as used in genetic engineering and fermentative production of chemical compounds by cultivation of microorganisms. See also Sambrook et al. (Sambrook, J. and Russell, D. W. Molecular cloning. A laboratory manual, 3rd ed, Cold Spring Harbor Laboratory Press, Cold Spring Harbor, N Y. 2001) and Chmiel et al. (Bioprocesstechnik 1. Einführung in die Bioverfahrenstechnik, Gustav Fischer Verlag, Stuttgart, 1991).

Electrocompetent Bacillus licheniformis Cells and Electroporation

Transformation of DNA into a Bacillus licheniformis strain as described in U.S. Pat. No. 5,352,604 is performed via electroporation. Preparation of electrocompetent Bacillus licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanperä J., 1989, FEMS Microbio. Lett., 61: 165-170) following plating on selective LB-agar plates.

In order to overcome the Bacillus licheniformis specific restriction modification system of Bacillus licheniformis strains, plasmid DNA is isolated from Ec #098 cells as described below.

### Plasmid Isolation

Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, N Y. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37 C prior to cell lysis.

### Molecular Biology Methods and Techniques

Standard methods in molecular biology not limited to cultivation of Bacillus and E. coli microorganisms, electroporation of DNA, isolation of genomic and plasmid DNA, PCR reactions, cloning technologies were performed as essentially described by Sambrook and Rusell. (Sambrook, J. and Russell, D. W. Molecular cloning. A laboratory manual, 3rd ed, Cold Spring Harbor Laboratory Press, Cold Spring Harbor, N Y. 2001.)

### Strains

E. coli Strain Ec #098

E. coli strain Ec #098 is an E. coli INV110 strain (Life technologies) carrying the DNA-methyltransferase encoding expression plasmid pMDS003 WO2019016051.

Generation of Bacillus licheniformis Gene k.o Strains

For gene deletion in a Bacillus licheniformis strain as described in U.S. Pat. No. 5,352,604 and derivatives thereof deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and analyzed for correctness by PCR analysis. The isolated plasmid DNA carries the DNA methylation pattern of Bacillus licheniformis as described in WO2019016051 and is protected from degradation upon transfer into Bacillus licheniformis.

aprE Gene Deletion Strain Bli #002

Electrocompetent Bacillus licheniformis cells as described in U.S. Pat. No. 5,352,604 were prepared as described above and transformed with 1 μg of pDel003 aprE gene deletion plasmid isolated from E. coli Ec #098 following plating on LB-agar plates containing 5 μg/ml erythromycin at 30° C.

The gene deletion procedure was performed as described in the following:

Plasmid carrying Bacillus licheniformis cells were grown on LB-agar plates with 5 μg/ml erythromycin at 45° C. forcing integration of the deletion plasmid via Campbell recombination into the chromosome with one of the homology regions of pDel003 homologous to the sequences 5′ or 3′ of the aprE gene. Clones were picked and cultivated in LB-media without selection pressure at 45° C. for 6 hours, following plating on LB-agar plates with 5 μg/ml erythromycin at 30° C. Individual clones were picked and analyzed by colony-PCR with oligonucleotides SEQ ID 06 and SEQ ID 07 for successful deletion of the aprE gene. Putative deletion positive individual clones were picked and taken through two consecutive overnight incubation in LB media without antibiotics at 45° C. to cure the plasmid and plated on LB-agar plates for overnight incubation at 30° C. Single clones were again restreaked on LB-agar plates with 5 μg/ml erythromycin and analyzed by colony PCR for successful deletion of the aprE gene. A single erythromycin-sensitive clone with the correct deleted aprE gene was isolated and designated Bli #002

amyB Gene Deletion Strain Bli #003

Electrocompetent Bacillus licheniformis Bli #002 cells were prepared as described above and transformed with 1 μg of pDel004 amyB gene deletion plasmid isolated from E. coli Ec #098 following plating on LB-agar plates containing 5 μg/ml erythromycin at 30° C.

The gene deletion procedure was performed as described for the aprE gene.

The deletion of the amyB gene was analyzed by PCR with oligonucleotides SEQ ID 09 and SEQ ID 10. The resulting Bacillus licheniformis strain with a deleted aprE and deleted amyB gene is designated Bli #003.

sigF Gene Deletion Strain Bli #004

Electrocompetent Bacillus licheniformis Bli #003 cells were prepared as described above and transformed with 1 μg of pDel005 sigF gene deletion plasmid isolated from E. coli Ec #098 following plating on LB-agar plates containing 5 μg/ml erythromycin at 30° C.

The gene deletion procedure was performed as described for the aprE gene.

The deletion of the sigF gene was analyzed by PCR with oligonucleotides SEQ ID 12 and SEQ ID 13 The resulting Bacillus licheniformis strain with a deleted aprE, a deleted amyB gene and a deleted sigF gene is designated Bli #004. Bacillus licheniformis strain Bli #004 is no longer able to sporulate as described (WO9703185).

### Poly-Gamma Glutamate Synthesis Genes Deletion Strain Bli #008

Electrocompetent Bacillus licheniformis Bli #004 cells were prepared as described above and transformed with 1 μg of pDel007 pga gene deletion plasmid isolated from E. coli Ec #098 following plating on LB-agar plates containing 5 μg/ml erythromycin at 30° C.

The gene deletion procedure was performed as described for the deletion of the aprE gene.

The deletion of the pga genes was analyzed by PCR with oligonucleotides SEQ ID 15 and SEQ ID 16 The resulting Bacillus licheniformis strain with a deleted aprE, a deleted amyB gene, a deleted sigF gene and a deleted pga gene cluster is designated Bli #008.

remA R18W P29S Strain Bli #030

Electrocompetent Bacillus licheniformis Bli #008 cells were prepared as described above and transformed with 1 μg of pDel034 remA gene editing plasmid isolated from E. coli Ec #098 following plating on LB-agar plates containing 5 μg/ml erythromycin at 30° C.

The gene deletion procedure was performed as described for the deletion of the aprE gene.

The gene editing of the remA gene was analyzed by PCR with oligonucleotides SEQ ID 18 and SEQ ID 19 following restriction enzyme cleavage with ClaI restriction endonuclease. The resulting Bacillus licheniformis strain with a deleted aprE, a deleted amyB gene, a deleted sigF gene, deleted pga gene cluster and mutated remA R18W P19S is designated Bli #030.

### Plasmids

pEC194RS—Bacillus Temperature Sensitive Deletion Plasmid.

The plasmid pE194 (Villafane, et al (1987): J. Bacteriol. 169(10), 4822-4829) is PCR-amplified with oligonucleotides SEQ ID 01 and SEQ ID 02 with flanking PvuII sites, digested with restriction endonuclease PvuII and ligated into vector pCE1 digested with restriction enzyme SmaI. pCE1 is a pUC18 derivative, where the BsaI site within the ampicillin resistance gene has been removed by a silent mutation. The ligation mixture was transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid is named pEC194S.

The type-II-assembly mRFP cassette is PCR-amplified from plasmid pBSd141R (accession number: KY995200) (Radeck, J., Mascher, T. 2017; Sci. Rep. 7: 14134) with oligonucleotides SEQ ID 03 and SEQ ID 04, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDel003—aprE Gene Deletion Plasmid

The gene deletion plasmid for the aprE gene of Bacillus licheniformis was constructed with plasmid pEC194RS and the gene synthesis construct SEQ ID 05 comprising the genomic regions 5′ and 3′ of the aprE gene flanked by BsaI sites compatible to pEC194RS. The type-II-assembly with restriction endonuclease BsaI was performed as described (Radeck et al., 2017) and the reaction mixture subsequently transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting aprE deletion plasmid is named pDel003.

pDel004—amyB Gene Deletion Plasmid

The gene deletion plasmid for the amyB gene of Bacillus licheniformis was constructed as described for pDel003, however the gene synthesis construct SEQ ID 08 comprising the genomic regions 5′ and 3′ of the amyB gene flanked by BsaI sites compatible to pEC 194RS was used. The resulting amyB deletion plasmid is named pDel004.

pDel005—sigF Gene Deletion Plasmid

The gene deletion plasmid for the sigF gene (spoIIAC gene) of Bacillus licheniformis was constructed as described for pDel003, however the gene synthesis construct SEQ ID 11 comprising the genomic regions 5′ and 3′ of the sigF gene flanked by BsaI sites compatible to pEC194RS was used. The resulting sigF deletion plasmid is named pDel005.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel003, however the gene synthesis construct SEQ ID 14 comprising the genomic regions 5′ and 3′ flanking the ywsC, ywtA (pgsC), ywtB (pgsA), ywtC (pgsE) genes flanked by BsaI sites compatible to pEC194RS was used. The resulting pga deletion plasmid is named pDel007.

pDel034—remA Loss of Function Plasmid

To inactivate RemA, the wildtype allele of Bacillus licheniformis was exchanged by a mutated copy of the remA gene at its native locus, resulting in expression of a RemA with the combined loss of function mutations R18W and P29S (Winkelman, J. T Kearns, D. B. (2009): Journal of bacteriology 191 (12), S. 3981-3991). The remA R18W, P29S gene with the 5′ and 3′ flanking regions flanked by BsaI sites compatible to pEC 194RS was ordered as gene synthesis construct SEQ ID 17. The gene editing plasmid was constructed as described for pDel003. The resulting remA editing plasmid was named pDel034.

### EXAMPLES

### Example 1: Identification of Conserved Amino Acid Position within RemA and RemB

Conserved positions of amino acids in a protein sequence of interest may be determined as follows:

In a first step, create a multiple sequence alignment with the sequence of interest and sequences from a database, preferably using program HHblits (preferably version 3.3.0) acting on the UniRef30 database (preferably version 2020_06) with using default parameters.

HHblits is part of the HH-suite (Steinegger M, Meier M, Mirdita M, Vöhringer H, Haunsberger S J, and Söding J (2019) HH-suite3 for fast remote homology detection and deep protein annotation, BMC Bioinformatics, 473) and can for example be downloaded from https://github.com/soedinglab/hh-suite/.

Database UniRef30 (Mirdita M, von den Driesch L, Galiez C, Martin M J, Söding J, Steinegger M. Uniclust databases of clustered and deeply annotated protein sequences and alignments. Nucleic Acids Res. 2017 Jan. 4; 45(D1): D170-D176.) can for example be downloaded from https://uniclust.mmseqs.com/.

To facilitate subsequent statistic calculations on each position in the alignment, the resulting alignment can also be converted to FASTA format. For example, the A3M alignment format can be converted to FASTA format with tool “reformat.pl”, which is also included within the HH-Suite, using the -r parameter.

In a second step, for each alignment position, the information content (IC) value then shall be computed as value R_Sequence (I) as is described by Schneider, T. D.; Stephens, R. M. Sequence Logos: A New Way to Display Consensus Sequences. Nucleic Acids Res. 1990, 18 (20), 6097-6100, with using 20 states for amino acid sequences.

A conserved position is defined as having an information content of 2.0 or higher.

Table 1 lists the IC values of the multiple sequence alignment (MAS) at the amino acid positions in reference to the query sequence of RemA (SEQ ID 21).

| | AA of SEQ | IC calculated | |
|---|---|---|---|
| | ID 21 at | from MAS at | |
| AA pos. of | given | given AA | |
| SEQ ID 21 | position | position | C |
| 1 | MET | 2.97 | * |
| 2 | THR | 0.94 | |
| 3 | ILE | 1.24 | |
| 4 | LYS | 1.72 | |
| 5 | LEU | 3.18 | * |
| 6 | ILE | 2.92 | * |
| 7 | ASN | 3.46 | * |
| 8 | ILE | 3.56 | * |
| 9 | GLY | 4.17 | * |
| 10 | PHE | 3.52 | * |
| 11 | GLY | 3.78 | * |
| 12 | ASN | 3.82 | * |
| 13 | ILE | 1.77 | |
| 14 | ILE | 3.48 | * |
| 15 | SER | 1.94 | |
| 16 | ALA | 2.35 | * |
| 17 | ASN | 1.78 | |
| 18 | ARG | 3.5 | * |
| 19 | LEU | 2.78 | * |
| 20 | ILE | 2.85 | * |
| 21 | SER | 2.91 | * |
| 22 | ILE | 3.33 | * |
| 23 | VAL | 3.08 | * |
| 24 | SER | 2.56 | * |
| 25 | PRO | 3.42 | * |
| 26 | GLU | 2.31 | * |
| 27 | SER | 3.66 | * |
| 28 | ALA | 3.43 | * |
| 29 | PRO | 3.84 | * |
| 30 | ILE | 2.5 | * |
| 31 | LYS | 3.75 | * |
| 32 | ARG | 3.98 | * |
| 33 | MET | 2.5 | * |
| 34 | ILE | 2.49 | * |
| 35 | GLN | 2.07 | * |
| 36 | ASP | 1.97 | |
| 37 | ALA | 3.4 | * |
| 38 | ARG | 3.03 | * |
| 39 | ASP | 2.03 | * |
| 40 | ARG | 1.34 | |
| 41 | GLY | 2.43 | * |
| 42 | MET | 1.15 | |
| 43 | LEU | 3.25 | * |
| 44 | ILE | 3.33 | * |
| 45 | ASP | 4.12 | * |
| 46 | ALA | 3.46 | * |
| 47 | THR | 4 | * |
| 48 | TYR | 2.01 | * |
| 49 | GLY | 4.26 | * |
| 50 | ARG | 3.89 | * |
| 51 | ARG | 3.14 | * |
| 52 | THR | 3.95 | * |
| 53 | ARG | 3.43 | * |
| 54 | ALA | 3.17 | * |
| 55 | VAL | 3.29 | * |
| 56 | VAL | 3.13 | * |
| 57 | ILE | 2.83 | * |
| 58 | MET | 2.58 | * |
| 59 | ASP | 3.53 | * |
| 60 | SER | 3.31 | * |
| 61 | ASP | 2.19 | * |
| 62 | HIS | 3.21 | * |
| 63 | ILE | 2.96 | * |
| 64 | ILE | 3.07 | * |
| 65 | LEU | 3.92 | * |
| 66 | SER | 3.79 | * |
| 67 | ALA | 3.28 | * |
| 68 | VAL | 2.45 | * |
| 69 | GLN | 2.56 | * |
| 70 | PRO | 2.96 | * |
| 71 | GLU | 3.48 | * |
| 72 | THR | 3.6 | * |
| 73 | VAL | 2.73 | * |
| 74 | ALA | 2.31 | * |
| 75 | GLN | 1.36 | |
| 76 | ARG | 3.95 | * |
| 77 | LEU | 1.99 | |
| 78 | SER | 0.54 | |
| 79 | VAL | 0.93 | |
| 80 | LYS | 0.57 | |
| 81 | GLU | 0.65 | |
| 82 | GLU | 0.59 | |
| 83 | ILE | 0.83 | |
| 84 | MET | 0.83 | |
| 85 | ASP | 1.71 | |
| 86 | GLU | 1.63 | |
| 87 | GLY | 1.45 | |
| 88 | GLN | 0 | |
| 89 | GLY | 0 | |

Pos. = position
AA = amino acid
IC = information content
C. = conserved amino acid with IC > 2.0; marked with * (asterix)
MAS. = multiple sequence alignment

Table 2 lists the IC values of the multiple sequence alignment (MAS) at the amino acid positions in reference to the query sequence of RemB (SEQ ID 23).

| | AA of SEQ | IC calculated | |
|---|---|---|---|
| | ID 23 at | from MAS at | |
| AA pos. of | given | given AA | |
| SEQ ID 23 | position | position | C |
| 1 | MET | 4.04 | * |
| 2 | TYR | 3.03 | * |
| 3 | ILE | 2.84 | * |
| 4 | HIS | 3.82 | * |
| 5 | LEU | 3.02 | * |
| 6 | GLY | 4.13 | * |
| 7 | ASP | 1.53 | |
| 8 | ASP | 2.23 | * |
| 9 | PHE | 0.9 | |
| 10 | VAL | 1.96 | |
| 11 | VAL | 2.98 | * |
| 12 | SER | 1.44 | |
| 13 | THR | 0.99 | |
| 14 | ARG | 1.5 | |
| 15 | GLU | 2.24 | * |
| 16 | ILE | 2.9 | * |
| 17 | VAL | 3.04 | * |
| 18 | ALA | 2.41 | * |
| 19 | ILE | 3.69 | * |
| 20 | PHE | 2.25 | * |
| 21 | ASP | 3.4 | * |
| 22 | TYR | 0.98 | |
| 23 | LYS | 1.33 | |
| 24 | ALA | 0.95 | |
| 25 | LYS | 0.85 | |
| 26 | THR | 0.82 | |
| 27 | SER | 2.74 | * |
| 28 | PRO | 1.01 | |
| 29 | ILE | 0.83 | |
| 30 | VAL | 1.58 | |
| 31 | GLU | 1.22 | |
| 32 | GLU | 1.29 | |
| 33 | PHE | 2.59 | * |
| 34 | LEU | 2.33 | * |
| 35 | SER | 0.79 | |
| 36 | LYS | 1.05 | |
| 37 | GLN | 1.35 | |
| 38 | LYS | 1.08 | |
| 39 | GLN | 1.58 | |
| 40 | ARG | 0.64 | |
| 41 | ILE | 2.11 | * |
| 42 | VAL | 1.2 | |
| 43 | SER | 0.6 | |
| 44 | SER | 1.52 | |
| 45 | ASN | 1.02 | |
| 46 | SER | 1.74 | |
| 47 | THR | 0.76 | |
| 48 | PRO | 1.52 | |
| 49 | LYS | 3.62 | * |
| 50 | SER | 3.59 | * |
| 51 | ILE | 1.9 | |
| 52 | VAL | 3.12 | * |
| 53 | VAL | 2.72 | * |
| 54 | THR | 2.81 | * |
| 55 | LEU | 0.77 | |
| 56 | GLN | 0.91 | |
| 57 | SER | 0.82 | |
| 58 | ILE | 2.62 | * |
| 59 | TYR | 3.61 | * |
| 60 | PHE | 1.68 | |
| 61 | SER | 3.88 | * |
| 62 | PRO | 2.35 | * |
| 63 | LEU | 2.86 | * |
| 64 | ALA | 3.4 | * |
| 65 | SER | 2.22 | * |
| 66 | GLY | 0.93 | |
| 67 | THR | 3.83 | * |
| 68 | LEU | 3.99 | * |
| 69 | LYS | 1.47 | |
| 70 | LYS | 3.11 | * |
| 71 | ARG | 4.18 | * |
| 72 | ALA | 1.66 | |
| 73 | GLN | 0.65 | |
| 74 | SER | 0.34 | |
| 75 | LYS | 0.49 | |
| 76 | PRO | 0.42 | |
| 77 | GLU | 0.62 | |
| 78 | ILE | 2.26 | * |
| 79 | ASP | 1.67 | |
| 80 | SER | 0 | |

### Example 2: Generation of B. licheniformis Enzyme Expression Strains

Bacillus licheniformis strains as listed in Table 3 were made competent as described above. Protease expression plasmid pUK56 (WO2019016051) was isolated from B. subtilis Bs #056 strain (WO2019016051) to carry the B. licheniformis specific DNA methylation pattern. Plasmids were transformed in the indicated strains and plated on LB-agar plates with 20 μg/μl kanamycin. Individual clones were analyzed for correctness of the plasmid DNA by restriction digest and functional enzyme expression was assessed by transfer of individual clones on LB-plates with 1% skim milk for clearing zone formation of protease producing strains. The resulting B. licheniformis expression strains are listed in Table 1.

TABLE 3
Overview on B. licheniformis expression strains

| B. licheniformis | Expression | B. licheniformis |
|---|---|---|
| Expression strain | plasmid | strain |
| BES#130 | pUK56 | Bli#008 |
| BES#131 | pUK56 | Bli#030 |

### Example 3: Cultivation of Bacillus licheniformis Protease Expression Strains

Bacillus licheniformis strains from Example 2 were cultivated in a fermentation process using a chemically defined fermentation medium.

The following macroelements were provided in the fermentation process:

| | | Concentration |
|---|---|---|
| Compound | Formula | [g/L initial volume] |
| Citric acid | C6H807 | 3.0 |
| Calcium sulfate | CaSO4 | 0.7 |
| Monopotassium phosphate | KH2PO4 | 25 |
| Magnesium sulfate | MgSO4*7H2O | 4.8 |
| Sodium hydroxide | NaOH | 4.0 |
| Ammonia | NH3 | 1.3 |

The following trace elements were provided in the fermentation process:

| Trace element | Symbol | Concentration [mM] |
|---|---|---|
| Manganese | Mn | 24 |
| Zinc | Zn | 17 |
| Copper | Cu | 32 |
| Cobalt | Co | 1 |
| Nickel | Ni | 2 |
| Molybdenum | Mo | 0.2 |
| Iron | Fe | 38 |

The fermentation was started with a medium containing 8 g/l glucose. A solution containing 50% glucose was used as feed solution. The pH was adjusted during fermentation using ammonia. In both experiments, the total amount of added chemically defined carbon source was kept above 200 g per liter of initial medium. Fermentations were carried out under aerobic conditions for a duration of more than 70 hours.

At the end of the fermentation process, samples were withdrawn and the protease activity determined photometrically: proteolytic activity was determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring at OD405.

The protease yield was calculated by dividing the product titer by the amount of glucose added per final reactor volume. The protease yield of strain BES #130 was set to 100% and the protease yield of the strain BFS #131 referenced to BES #130 accordingly (Table 4). B. licheniformis expression strain BES #131, with the mutated remA gene (resulting in an altered RemA protein comprising the mutations R18W und P29S) showed 10% improvement in the protease yield compared to B. licheniformis expression strain BES #130.

TABLE 4
Protease yield of Bacillus licheniformis expression strains

| B. licheniformis Expression strain | Protease yield [%] |
|---|---|
| BES#130 | 100 |
| BES#131 | 110 |

TABLE 5
Gene names, protein names and sequences
of RemA and RemB of different organisms

| | Gene | | Protein | | |
|---|---|---|---|---|---|
| Organism | Name | Synonym | Name | DNA | PRT |
| Bacillus licheniformis | ylzA | remA | RemA | SEQ ID 20 | SEQ ID 21 |
| Bacillus subtilis | ylzA | remA | RemA | SEQ ID 24 | SEQ ID 25 |
| Bacillus pumilus | ylzA | remA | RemA | SEQ ID 28 | SEQ ID 29 |
| Bacillus velezensis | ylzA | remA | RemA | SEQ ID 32 | SEQ ID 33 |
| Bacillus | ylzA | remA | RemA | SEQ ID 36 | SEQ ID 37 |
| amyloliquefaciens | | | | | |
| Bacillus licheniformis | yaaB | remB | RemB | SEQ ID 22 | SEQ ID 23 |
| Bacillus subtilis | yaaB | remB | RemB | SEQ ID 26 | SEQ ID 27 |
| Bacillus pumilus | yaaB | remB | RemB | SEQ ID 30 | SEQ ID 31 |
| Bacillus velezensis | yaaB | remB | RemB | SEQ ID 34 | SEQ ID 35 |
| Bacillus | yaaB | remB | RemB | SEQ ID 38 | SEQ ID 39 |
| amyloliquefaciens | | | | | |

1. A modified Bacillus host cell comprising an altered RemA protein and/or an altered RemB protein, wherein
i) the altered RemA protein comprises a nonsense mutation in the gene coding for the RemA protein and/or one or more missense mutations in the gene coding for the RemA protein at positions coding for conserved amino acids in the RemA protein at one or more of amino acid positions corresponding to amino acid positions 5-77 of SEQ ID NO: 21,
ii) the altered RemB protein comprises a nonsense mutation in the gene coding for the RemB protein and/or one or more missense mutations in the gene coding for the RemB protein at positions coding for conserved amino acids in the RemB protein at one or more of amino acid positions corresponding to amino acid positions 4-71 of SEQ ID NO: 23, and/or
iii) the altered RemA and/or RemB protein is an inactivated RemA and/or RemB protein,
wherein the Bacillus host cell is not a Bacillus subtilis cell.

2. The modified Bacillus host cell of claim 1, wherein the one or more mutations in the gene coding for the RemA protein result in non-conservative amino acid substitutions at conserved amino acid positions of SEQ ID NO: 21, 25, 29, 33, or 37 with an IC value equal or greater than 3.0.

3. The modified Bacillus host cell of claim 1, wherein the one or more mutations in the gene coding for the RemB protein result in non-conservative amino acid substitutions at conserved amino acid positions of SEQ ID NO: 23, 27, 31, 35, or 39 with an IC value equal or greater than 3.0.

4. The modified Bacillus host cell of claim 1, wherein the host cell belongs to the species Bacillus alcalophilus, Bacillus amyloliquefaciens, Bacillus brevis, Bacillus cereus, Bacillus circulans, Bacillus clausii, Bacillus coagulans, Bacillus firmus, Bacillus globigii, Bacillus halodurans, Bacillus lautus, Bacillus lentus, Bacillus licheniformis, Bacillus paralicheniformis, Bacillus megaterium, Bacillus methanolicus, Bacillus methylotrophicus, Bacillus mojavensis, Bacillus pumilus, Geobacillus stearothermophilus (Bacillus stearothermophilus), Bacillus thuringiensis or Bacillus velezensis, preferably Bacillus licheniformis.

5. The modified Bacillus host cell of claim 1, wherein the altered RemA protein has at least 80%, but below 100% sequence identity to SEQ ID NO: 21, 25, 29, 33, or 37, and wherein the altered RemB protein has at least 80%, but below 100% sequence identity to SEQ ID NO: 23, 27, 31, 35, or 39.

6. The modified Bacillus host cell of claim 1, wherein the host cell comprises an expression cassette for the production of a compound of interest.

7. The modified Bacillus host cell of claim 6, wherein the compound of interest is an enzyme selected from the group consisting of amylase, protease, lipase, phospholipase, mannanase, phytase, xylanase, lactase, phosphatase, glucoamylase, nuclease, galactosidase, endoglucanase and cellulase.

8. The modified Bacillus host cell of claim 6, wherein the modified Bacillus host cell comprises an increased production of the compound of interest compared to a Bacillus control cell that does not comprise the altered RemA protein and/or the altered RemB protein.

9. A method for producing a compound of interest, preferably a polypeptide of interest, comprising
a) providing a modified Bacillus host cell as defined in claim 1,
b) cultivating the host cell under conditions which allow for the expression of the compound of interest, and
c) optionally isolating the compound of interest from the cultivation medium.

10. The method for producing a compound of interest of claim 8, wherein the Bacillus host cell is selected from the group consisting of Bacillus pumilus, Bacillus velezensis, Bacillus amyloliquefaciens, Bacillus alcalophilus, Bacillus licheniformis, Bacillus paralicheniformis, Bacillus lentus, Bacillus clausii, Bacillus halodurans, Bacillus megaterium, Bacillus methanolicus, Geobacillus stearothermophilus (Bacillus stearothermophilus), Bacillus mojavensis, Bacillus globigii, and Bacillus subtilis, preferably Bacillus licheniformis.

11. The method of claim 9, wherein the expression of the compound of interest is increased as compared to the expression of the compound of interest in a Bacillus control cell that does not comprise the altered RemA protein and/or the altered RemB protein.

12. An altered RemA protein, wherein the altered RemA protein comprises one or more non-conservative amino acid substitutions at conserved amino acid positions of SEQ ID NO: 21 with an IC value equal or greater than 3.0, wherein the altered RemA protein has at least 97%, at least 98%, or at least 99%, but less than 100% sequence identity to SEQ ID NO: 21.

13. An altered RemB protein, wherein the altered RemB protein comprises one or more non-conservative amino acid substitutions at conserved amino acid positions of SEQ ID NO: 23, 27, 31, 35, or 39 with an IC value equal or greater than 3.0,
wherein the altered RemB protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 23.

14. The modified Bacillus host cell of claim 1, wherein the modified Bacillus host cell is a Bacillus licheniformis host cell.

15. (canceled)

16. (canceled)

17. (canceled)

18. The modified Bacillus host cell of claim 1, wherein the one or more mutations in the gene coding for the RemA protein result in non-conservative amino acid substitutions at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21.

19. The modified Bacillus host cell of claim 1, wherein the one or more mutations in the gene coding for the RemB protein result in non-conservative amino acid substitutions at one or more amino acid positions corresponding to amino acid positions selected from the group consisting of H4, G6, I19, K49, S50, Y59, S61, T67, L68, and R71 of SEQ ID NO: 23.

20. An altered RemA protein, wherein the altered RemA protein comprises one or more non-conservative amino acid substitutions at one or more amino acid positions corresponding to amino acid positions selected from the group consisting of I8, G9, F10, G11, N12, R18, S27, P29, K31, R32, D45, T47, G49, R50, T52, D59, L65, S66, T72, and R76 of SEQ ID NO: 21, wherein the altered RemA protein has at least 97%, at least 98%, or at least 99%, but less than 100% sequence identity to SEQ ID NO: 21.

21. An altered RemB protein, wherein the altered RemB protein comprises one or more non-conservative amino acid substitutions at one or more amino acid positions corresponding to amino acid positions selected from the group consisting of H4, G6, I19, K49, S50, Y59, S61, T67, L68, and R71 of SEQ ID NO: 23,
wherein the altered RemB protein has at least 80%, at least 85%, at least 90%, at least 91%, at least 92%, at least 93%, at least 94%, at least 95%, at least 96%, at least 97%, at least 98%, or at least 99%, but less than 100% sequence identity to SEQ ID NO: 23.


SEQUENCE LISTING (49 sequences)

SEQ ID NO: 1 - DNA, 32 bp, Artificial sequence
  Oligonucleotide PCR-amplification of pE194
tatatacagctggattcacaaaaaataggcac

SEQ ID NO: 2 - DNA, 33 bp, Artificial Sequence
  Oligonucleotide PCR-amplification of pE194
tatatacagctggattatgtcttttgcgcagtc

SEQ ID NO: 3 - DNA, 30 bp, Artificial Sequence
  Oligonucleotide PCR-amplification of functional mRFP cassette
tatatggatccgtaatcagggtatcgaggc

SEQ ID NO: 4 - DNA, 31 bp, Artificial Sequence
  Oligonucleotide PCR-amplification of functional mRFP cassette
tatatggatccctcattaggcgggctactaa

SEQ ID NO: 5 - DNA, 1027 bp, Artificial Sequence
  Homology region of the fusion of 5-prime and 3-prime regions of aprE gene with flanking BsaI sites
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 6 - DNA, 21 bp, Artificial Sequence
  Oligonucleotide PCR-amplification of aprE genomic region
ccggttgtcattgatccttta

SEQ ID NO: 7 - DNA, 21 bp, Artificial Sequence
  Oligonucleotide PCR-amplification of aprE genomic region
atcctcctgcaaaaaccgtat

SEQ ID NO: 8 - DNA, 1022 bp, Artificial Sequence
  Homology region of the fusion of 5-prime and 3-prime regions of amyB gene with flanking BsaI sites
ggtctcgacccacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtatttgttaaaaattcggaatatttatacaatatcatatgtttcacattgaaaggggaggagaatctagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattatctcaggagacc

SEQ ID NO: 9 - DNA, 22 bp, Artificial Sequence
  Oligonucleotide PCR-amplification of amyB genomic region
agaagcatgaagggcatgcgac

SEQ ID NO: 10 - DNA, 21 bp, Artificial Sequence
  Oligonucleotide PCR-amplification of amyB genomic region
caacaacaggctgtctgacgg

SEQ ID NO: 11 - DNA, 1022 bp, Artificial Sequence
  Homology region of the fusion of 5-prime and 3-prime regions of sigF gene with flanking BsaI sites
ggtctcgacccaaaatcattcgccttgagcaatcagagcagcgtgcacttgaaacgttgggggtggcgtcatgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctgatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaatgaggctgctcatgttgcaggcagcctcggatgtccgatgaaaaaccggacgctcttgggagcgttccggttttttttgtgtggtaatttatggtcttttgcgcctttctgaatgataaatgggatgtacttcatactacaactataaccatcatataggaagtgacccagatggaacgtcaagtttttatcagactccgccaccggcttgaggcagatccggatgaattgattttgctcggccatatcgcacaggtagcgggtgaccgcggatacaaagaaaagcttgaacggctgcctatttatcaggtcagcaaagcggatcaaagcatggtggtgctggacgtgatgaaggtgattgaagctgtacataaatcgtttcctgaccttgatgtccaaaccgtcggcggttctgaaaccatcgtggagatccaatatccgaaaaggggtctgtcgcccgtgcttttcatcgccgtctggctgctcttgttcgtcggtgcctcaggagacc

SEQ ID NO: 12 - DNA, 19 bp, Artificial Sequence
  Oligonucleotide PCR-amplification of sigF genomic region
agcagctcggcggagaaat

SEQ ID NO: 13 - DNA, 21 bp, Artificial Sequence
  Oligonucleotide PCR-amplification of sigF genomic region
accttgcccgtcaccatttcg

SEQ ID NO: 14 - DNA, 1363 bp, Artificial Sequence
  Homology region of the fusion of 5-prime and 3-prime regions of pga genes with flanking BsaI sites
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 15 - DNA, 20 bp, Artificial Sequence
  Oligonucleotide PCR-amplification of pga genomic region
aaagccttctcctctctatt

SEQ ID NO: 16 - DNA, 20 bp, Artificial Sequence
  Oligonucleotide PCR-amplification of pga genomic region
ttcttgaaaaagacaaggtc

SEQ ID NO: 17 - DNA, 794 bp, Artificial Sequence
  Homology regions of gene synthesis HomA + HomB remA R18W, P29S
ggtggtctctacccaagtcgcactgtttgcagaccgttcggatatcacggaagaagtgacgaggctgaaaagccatttccgccagttccgcgatatttgcaaagcgggaggagccgcggggagaaagctcgatttcctcgtccaggagctcaaccgtgaagcgaacacgatcggttcaaaagcgaatgatcaccagatcacaaaacatgtggtcgaaatgaaaagctctattgaaaaaataaaagaacaagtgcaaaatatagaatagcgattgtgcgtattgtttacggatgttctctgcaggttaaactagagacgtccaagtacagggggaacgtataggatgacgattaaactgatcaatatcggctttggaaatatcatatccgcgaattggctgatctcgattgtgagtcctgagtccgcatcgattaagcggatgatccaggatgcccgcgacagaggcatgcttatagatgctacatatggaagaagaacccgtgcggttgtcattatggacagtgaccatatcatcttatctgccgtccagcctgagacagtagcacaaaggctttccgttaaagaagaaattatggatgaagggcagggataagagctttatgaaagaaagaggtttgttaatcgttctctccggcccttccggcgtcggaaaaggaacagtcaggcaggcgctgtttgctcaggaggacacaaaatttgaatattcgatttcggtgacgacaagaaaaccgcgtcaaggcgaaagagacggcgtcgactctcaagagaccacc

SEQ ID NO: 18 - DNA, 24 bp, Artificial Sequence
  Oligonucleotide PCR-amplification of remA genomic region
tccaggttgactggccgctgcttg

SEQ ID NO: 19 - DNA, 24 bp, Artificial Sequence
  Oligonucleotide PCR-amplification of remA genomic region
tttcaattcggcgaggctcggagg

SEQ ID NO: 20 - DNA, 270 bp, Bacillus licheniformis
  CDS (1)..(270): CDS of yzlA (remA), strain ATCC 14580
atgacgattaaactgatcaatatcggctttggaaatatcatatccgcgaatcggctgatctcgattgtgagtcctgagtccgcgcctattaagcggatgatccaggatgccagagaccgcggcatgcttatagatgctacatatggaagaagaacccgtgcggttgtcattatggacagtgaccatatcatcttatctgccgtccagcctgagacagtagcacaaaggctttccgttaaagaagaaattatggatgaagggcagggataa

SEQ ID NO: 21 - protein, 89 aa, Bacillus licheniformis
MTIKLINIGFGNIISANRLISIVSPESAPIKRMIQDARDRGMLIDATYGRRTRAVVIMDSDHIILSAVQPETVAQRLSVKEEIMDEGQG

SEQ ID NO: 22 - protein, 89 aa, Bacillus licheniformis; identical to SEQ ID NO: 21
  MISC_FEATURE (1)..(89): Protein of RemA, strain ATCC 14580

SEQ ID NO: 23 - DNA, 243 bp, Bacillus licheniformis
  CDS (1)..(243): CDS of yaaB (remB), strain ATCC 14580
ttgtatatccacttaggtgacgattttgtcgtctcaacgcgtgaaattgtggctatttttgattacaaggcaaagacatcgccgattgttgaggagtttttaagcaagcaaaaacagcggattgtctcttctaacagcacgccgaagtcaattgttgtcacattacaatcgatttatttttctcctttagcctcaggcacgttgaaaaaacgggcgcaatccaagccggaaatcgattcataa

SEQ ID NO: 24 - protein, 80 aa, Bacillus licheniformis
LYIHLGDDFVVSTREIVAIFDYKAKTSPIVEEFLSKQKQRIVSSNSTPKSIVVTLQSIYFSPLASGTLKKRAQSKPEIDS

SEQ ID NO: 25 - protein, 80 aa, Bacillus licheniformis
  MISC_FEATURE (1)..(80): Protein of RemB, strain ATCC 14580
MYIHLGDDFVVSTREIVAIFDYKAKTSPIVEEFLSKQKQRIVSSNSTPKSIVVTLQSIYFSPLASGTLKKRAQSKPEIDS

SEQ ID NO: 26 - DNA, 270 bp, Bacillus subtilis
  CDS (1)..(270): CDS of ylzA (remA)
atgacgattaaactgattaatatcggatttggcaatatcatctccgccaatcggatgatttcgattgtcagcccggagtctgcgccaatcaaacggatgattcaggatgcaagagaccgcggaatgctaattgacgctacatacggacgaagaacccgtgcagttgtcgtcatggatagtgatcacattatcttatctgccgtccagcctgagacagttgcacacagactttctgttaaagaagaaattatggatgaagggcaggggtaa

SEQ ID NO: 27 - protein, 89 aa, Bacillus subtilis
MTIKLINIGFGNIISANRMISIVSPESAPIKRMIQDARDRGMLIDATYGRRTRAVVVMDSDHIILSAVQPETVAHRLSVKEEIMDEGQG

SEQ ID NO: 28 - protein, 89 aa, Bacillus subtilis; identical to SEQ ID NO: 27
  MISC_FEATURE (1)..(89): Protein of RemA

SEQ ID NO: 29 - DNA, 246 bp, Bacillus subtilis
  CDS (1)..(246): CDS of yaaB (remB)
ttgtatattcatttaggtgatgactttgtggtttcaacacgagatattgtcggcatttttgactttaaagccaacatgtcgcctattgttgaagaatttctgaaaaaacagaaacacaaggtggtgccttccgtaaacggcacgcccaaatctatcgtagtcacggttcagaatatatattactctcccttatcttccagcacattaaaaaaacgtgcgcaatttatgtttgaaatagattcttag

SEQ ID NO: 30 - protein, 81 aa, Bacillus subtilis
LYIHLGDDFVVSTRDIVGIFDFKANMSPIVEEFLKKQKHKVVPSVNGTPKSIVVTVQNIYYSPLSSSTLKKRAQFMFEIDS

SEQ ID NO: 31 - protein, 81 aa, Bacillus subtilis
  MISC_FEATURE (1)..(81): Protein of Rem B
MYIHLGDDFVVSTRDIVGIFDFKANMSPIVEEFLKKQKHKVVPSVNGTPKSIVVTVQNIYYSPLSSSTLKKRAQFMFEIDS

SEQ ID NO: 32 - DNA, 270 bp, Bacillus pumilus
  CDS (1)..(270): CDS of ylzA (remA)
atgaccataaagttgatcaatatcggatttggaaacatcatttcagcgaatcggatgatttcgatcgtcagtccggaatcagcaccgattaaacgaatgatacaagacgcaagagatcgaggcatgctcatagatgctacttacggaagaagaacccgtgctgttgtcattatggacagtgaccatgtcatcttatctgccgttcagcctgagactgtcgcgcaaagactttctgtaaaagaagaaatcatagatgaagggcaggggtaa

SEQ ID NO: 33 - protein, 89 aa, Bacillus pumilus
MTIKLINIGFGNIISANRMISIVSPESAPIKRMIQDARDRGMLIDATYGRRTRAVVIMDSDHVILSAVQPETVAQRLSVKEEIIDEGQG

SEQ ID NO: 34 - protein, 89 aa, Bacillus pumilus; identical to SEQ ID NO: 33
  MISC_FEATURE (1)..(89): Protein of RemA

SEQ ID NO: 35 - DNA, 255 bp, Bacillus pumilus
  CDS (1)..(255): CDS of yaaB (remB)
gtgtgggtcttgtatattcatttaggtgatgattgtgttgtttctacacgagagattgtcgcaattgttgattacaaaatgaggtcgtcttctgttgtagaagagtttcttcaaaaacaagaaggacaaatcatttcgttatcacaagggacacccaaatccatcgtcgtcacaactaaatctgtttattactctcctctttcctcaagcacgctcaaaaaacgtgcttcatttgtgattgaaattgaagtctaa

SEQ ID NO: 36 - protein, 84 aa, Bacillus pumilus
VWVLYIHLGDDCVVSTREIVAIVDYKMRSSSVVEEFLQKQEGQIISLSQGTPKSIVVTTKSVYYSPLSSSTLKKRASFVIEIEV

SEQ ID NO: 37 - protein, 84 aa, Bacillus pumilus
  MISC_FEATURE (1)..(84): Protein of RemB
MWVLYIHLGDDCVVSTREIVAIVDYKMRSSSVVEEFLQKQEGQIISLSQGTPKSIVVTTKSVYYSPLSSSTLKKRASFVIEIEV

SEQ ID NO: 38 - DNA, 270 bp, Bacillus velezensis sp.
  CDS (1)..(270): CDS of ylzA (remA)
atgacgattaaactgattaatatcggatttggcaatatcatctccgccaatcggatgatttcgattgtcagcccggagtctgcgccaataaagcgaatgatccaggatgcaagagaccgcggaatgctaatagacgctacatacggacgaagaacccgtgcagttgtcgtcatggatagtgatcacattatcttatctgccgtccagcctgagacagttgcacacagactttctgtgaaagaagaaattatggatgaagggcaggggtaa

SEQ ID NO: 39 - protein, 89 aa, Bacillus velezensis sp.; identical to SEQ ID NO: 27

SEQ ID NO: 40 - protein, 89 aa, Bacillus velezensis sp.; identical to SEQ ID NO: 27
  MISC_FEATURE (1)..(89): Protein of RemA

SEQ ID NO: 41 - DNA, 246 bp, Bacillus velezensis sp.
  CDS (1)..(246): CDS of yaaB (remB)
ttgtatattcatttaggtgacgactttgtcgtttcaacacgcgatatcgtcggcatttttgactgcaaggtcaatgtatcgccgattgttgaagaatttctggacagacagaaagaaaaagtagtgccgtccgtaaacggcacgccaaagtctatcgtagtcacgactgagaatatatattactctcccttatcttccggcacactgaagaaacgtgcgcaatttatgttagaaatagattcttag

SEQ ID NO: 42 - protein, 81 aa, Bacillus velezensis sp.
LYIHLGDDFVVSTRDIVGIFDCKVNVSPIVEEFLDRQKEKVVPSVNGTPKSIVVTTENIYYSPLSSGTLKKRAQFMLEIDS

SEQ ID NO: 43 - protein, 81 aa, Bacillus velezensis sp.
  MISC_FEATURE (1)..(81): Proteoin of RemB
MYIHLGDDFVVSTRDIVGIFDCKVNVSPIVEEFLDRQKEKVVPSVNGTPKSIVVTTENIYYSPLSSGTLKKRAQFMLEIDS

SEQ ID NO: 44 - DNA, 270 bp, Bacillus amyloliquefaciens
  CDS (1)..(270): CDS of ylzA (remA)
atgacgattaaactgattaatatcggatttggcaatatcatctccgccaatcggatgatttcgattgtcagcccggagtctgcgccaataaagcgaatgattcaggatgcaagagaccgcggaatgctaatagacgctacatacggacgaagaacccgtgcagttgtcgtcatggatagtgatcacattatcttatctgccgtccagcctgagacagttgcacacagactttctgtgaaaggagaaattatggatgaagggcaggggtaa

SEQ ID NO: 45 - protein, 89 aa, Bacillus amyloliquefaciens
MTIKLINIGFGNIISANRMISIVSPESAPIKRMIQDARDRGMLIDATYGRRTRAVVVMDSDHIILSAVQPETVAHRLSVKGEIMDEGQG

SEQ ID NO: 46 - protein, 89 aa, Bacillus amyloliquefaciens; identical to SEQ ID NO: 45
  MISC_FEATURE (1)..(89): Protein of RemA

SEQ ID NO: 47 - DNA, 246 bp, Bacillus amyloliquefaciens
  CDS (1)..(246): CDS of yaaB (remB)
ttgtatattcatttaggtgacgactttgtcgtttcaacacgcgatatcgtcggcatttttgactgcaaggtcaatgtatcgccgatcgttgaagaatttctggacagacagaaagaaaaagtagtgccgtccgtaaatggcacaccaaagtctatcgtagtcacgactgagaatatatattactctcccttatcttccggcacactgaagaaacgtgcgcaatttatgttagaaatagattcttag

SEQ ID NO: 48 - protein, 81 aa, Bacillus amyloliquefaciens; identical to SEQ ID NO: 42

SEQ ID NO: 49 - protein, 81 aa, Bacillus amyloliquefaciens; identical to SEQ ID NO: 43
  MISC_FEATURE (1)..(81): Protein of RemB